SEQUENCE LISTING (26 sequences)

SEQ ID NO: 1 - DNA, 30 bp, Artificial Sequence
  Description of Artificial Sequence Synthetic
gatcgggaaagggaaaccgaaactgaagcc

SEQ ID NO: 2 - DNA, 30 bp, Artificial Sequence
  Description of Artificial Sequence Synthetic
gatcggcttcagtttcggtttccctttccc

SEQ ID NO: 3 - protein, 5 aa, Artificial Sequence
  Description of Artificial Sequence Synthetic
DYTDE

SEQ ID NO: 4 - protein, 5 aa, Artificial Sequence
  Description of Artificial Sequence Synthetic
AYTAA

SEQ ID NO: 5 - protein, 5 aa, Artificial Sequence
  Description of Artificial Sequence Synthetic
ELKNH

SEQ ID NO: 6 - protein, 5 aa, Artificial Sequence
  Description of Artificial Sequence Synthetic
ALANA

SEQ ID NO: 7 - protein, 6 aa, Artificial Sequence
  Description of Artificial Sequence Synthetic
KPEDLK

SEQ ID NO: 8 - protein, 6 aa, Artificial Sequence
  Description of Artificial Sequence Synthetic
APAALA

SEQ ID NO: 9 - protein, 5 aa, Artificial Sequence
  Description of Artificial Sequence Synthetic
DLTDE

SEQ ID NO: 10 - protein, 5 aa, Artificial Sequence
  Description of Artificial Sequence Synthetic
ALTAA

SEQ ID NO: 11 - protein, 5 aa, Artificial Sequence
  Description of Artificial Sequence Synthetic
RSTHE

SEQ ID NO: 12 - protein, 5 aa, Artificial Sequence
  Description of Artificial Sequence Synthetic
ASTAA

SEQ ID NO: 13 - protein, 5 aa, Artificial Sequence
  Description of Artificial Sequence Synthetic
DMSFE

SEQ ID NO: 14 - protein, 5 aa, Artificial Sequence
  Description of Artificial Sequence Synthetic
AMSFA

SEQ ID NO: 15 - protein, 7 aa, Artificial Sequence
  Description of Artificial Sequence Synthetic
EEELQFD

SEQ ID NO: 16 - protein, 7 aa, Artificial Sequence
  Description of Artificial Sequence Synthetic
AAALQFA

SEQ ID NO: 17 - protein, 5 aa, Artificial Sequence
  Description of Artificial Sequence Synthetic
EEQSE

SEQ ID NO: 18 - protein, 5 aa, Artificial Sequence
  Description of Artificial Sequence Synthetic
AAQSA

SEQ ID NO: 19 - protein, 5 aa, Artificial Sequence
  Description of Artificial Sequence Synthetic
KKHKP

SEQ ID NO: 20 - protein, 5 aa, Artificial Sequence
  Description of Artificial Sequence Synthetic
AAHAP

SEQ ID NO: 21 - protein, 5 aa, Artificial Sequence
  Description of Artificial Sequence Synthetic
EIKGN

SEQ ID NO: 22 - protein, 5 aa, Artificial Sequence
  Description of Artificial Sequence Synthetic
AIAGN

SEQ ID NO: 23 - protein, 6 aa, Artificial Sequence
  Description of Artificial Sequence Synthetic
EHSDEA

SEQ ID NO: 24 - protein, 6 aa, Artificial Sequence
  Description of Artificial Sequence Synthetic
AASAAQ

SEQ ID NO: 25 - DNA, 6152 bp, Artificial Sequence
  Description of Artificial Sequence Synthetic
gaattcctaaaaatagcaaagatgcttttgagccagaatgccttcatcgtcagatcacttaatttggttctcatggtgtatatcagcctcgtgtttggtatttcatatgattcgcctgattacacagatgaatcttgcactttcaagatatcattgcgaaatttccggtccatcttatcatgggaattaaaaaaccactccattgtaccaactcactatacattgctgtatacaatcatgagtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacattaatgtgatggtgaaatttccatctattgttgaggaagaattacagtttgatttatctctcgtcattgaagaacagtcagagggaattgttaagaagcataaacccgaaataaaaggaaacatgagtggaaatttcacctatatcattgacaagttaattccaaacacgaactactgtgtatctgtttatttagagcacagtgatgagcaagcagtaataaagtctcccttaaaatgcaccctccttccacctggccaggaatcagaatcagcagaatctgccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgagtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcgacctgcagaagcttagaaccgaggggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggctcgttagaacgcggctacaattaatacataaccttatgtatcatacacatacgatttaggtgacactatagaataacatccactttgcctttctctccacaggtgtccactcccaggtccaactgcaggccatggcggccatcgatt

SEQ ID NO: 26 - protein, 443 aa, Artificial Sequence
  Description of Artificial Sequence Synthetic
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2003/0018174 A1
(43) Pub. Date: Jan. 23, 2003

(54) Monoclonal antibodies to IFNAR2

(75) Inventors: Kyung Jin Kim, Los Altos, CA (US); Anan Chuntharapai, Colma, CA (US); Ji Lu, Fremont, CA (US)

Correspondence Address: Richard F. Trecartin, Esq., FLEHR HOHBACH TEST ALBRITTON & HERBERT LLP, Suite 3400, Four Embarcadero Center, San Francisco, CA, 94111-4187, US

(73) Assignee: Genentech, Inc.

(21) Appl. No.: 10/053,302

(22) Filed: Jan. 17, 2002

Related U.S. Application Data
(63) Continuation of application No. 09/166,298, filed on Oct. 5, 1998, now abandoned.
(60) Provisional application No. 60/061,185, filed on Oct. 6, 1997.

(51) Int. Cl. C07K 16/24; C07K 16/42

(52) U.S. Cl. 530/388.23; 530/387.2

(57) ABSTRACT

Anti-INFAR2 monoclonal antibodies with blocking antibodies against the INFAR2 binding of various type I interferons are provided.

### FIELD OF THE INVENTION;

This invention relates to the field of anti-type I interferon receptor antibodies, and more particularly to anti-type I interferon receptor antibodies that block the binding of type I interferons to the second component (IFNAR2) of the type I interferon receptor complex.

### BACKGROUND OF THE INVENTION

The type I interferons (IFNs) are cytokines which have pleiotropic effects on a wide variety of cell types. IFNs are best known for their anti-viral activity, but they also have anti-bacterial, anti-protozoal, immunomodulatory, and cell-growth regulatory functions. The type 1 interferons include interferon-α (IFN-α) and interferon-β (IFN-β). Human IFN-α (hIFN-α) is a heterogeneous family with at least 23 polypeptides while there is only one IFN-β polypeptide (J. Interferon Res., 13: 443444 (1993)). The hIFN-α subtypes show more than 70% amino acid sequence homology, and there is approximately 25% amino acid identity with hIFN-β. The hIFNs-α and hIFN-β share a common receptor.

Two components of the hIFN-α receptor complex have recently been cloned. The CDNA for the first hIFN-α receptor (hIFNAR1) encodes a 63 kD receptor protein (reported in Uze et al., Cell, 60: 225-234 (1990)). This receptor undergoes extensive glycosylation which causes it to migrate in gel electrophoresis as a much larger 135 kD protein. The second interferon receptor, hIFNAR2 (hIFN-αβR long), is a 115 kD protein which mediates a functional signaling complex when associated with hIFNAR1 (reported in Domanski et al., J. Biol Chem., 270: 21606-21611 (1995)). A variant of IFNAR2, the IFN-α/β receptor (HIFN-αβR short), is a 55 kD protein that can bind to type 1 hIFNs but cannot form a functional complex when associated with HIFNAR1 (reported in Novick et al., Cell, 77: 391400 (1994)). This IFN-α/β receptor appears to be an alternatively spliced variant of hIFNAR2.

The unprocessed hIFNAR1 expression product is composed of 557 amino acids including an extracellular domain (ECD) of 409 residues, a transmembrane domain of 21 residues, and an intracellular domain of 100 residues as shown in FIG. 5 on page 229 of Uze et al., supra. The ECD of IFNAR1 is composed of two domains, domain 1 and domain 2, which are separated by a three-proline motif. There is 19% sequence identity and 50% sequence homology between domains 1 and 2 (Uze et al., supra). Each domain (D200) is composed of approximately 200 residues and can be further subdivided into two homologous subdomains (SD100) of approximately 100 amino acids. The unprocessed hIFNAR2 expression product is composed of 515 amino acids, including an extracellular domain (ECD) of 217 residues, a transmembrane domain of 21 residues, and a long cytoplasmic tail of 250 residues as shown in FIG. 1 on page 21608 of Domanski et al., J. Biol. Chem., 37: 21606-21611 (1995).

Through the use of IFNAR1 gene knockout mice, IFNAR1 has been shown to be essential for the response to all type 1 IFNs (Muller et aL, Science, 264: 1918-1921 (1994); Cleary et al., J. Biol Chem., 269: 18747-18749 (1994)) and for the mediation of species-specific IFN signal transduction (Constantinescu et al., Proc. NatL. Acad. Sci. USA, 91: 9602-9606 (1994)). However, IFNAR2, not IFNAR1, plays a crucial role in ligand binding (Cohen et al., Mol. Cell Biol., 15: 4208 (1995)).

Benoit et al., J. Immunol., 150: 707-716 (1993) reported an anti-IFNAR1 mAb, 64G12, that was found to inhibit the binding of IFN-α2 (IFN-αA) and IFN-αB to Daudi cells and to inhibit the antiviral activity of IFN-α2, IFN-β and IFN-ω (IFN-α_{II}1) on Daudi cells. Benoit et al. also reported that 64G12 recognizes an epitope present in domain 1 of IFNAR1. Eid and Tovey, J. Interferon Cytokine Res., 15: 205-211 (1995) reported that 64G12 cannot immunoprecipitate cross-linked IFN-α2-receptor complexes from Daudi cells.

Colamonici and Domanski, J. Biol. Chem., 268: 10895-10899 (1993) reported an anti-IFNAR2 mAb (denoted the “IFNaRβ1 mAb”) that blocked the binding of IFN-α2 (IFN-αA) to Daudi cells and U-266 cells and blocked the antiproliferative activity of different type I interferons on Daudi cells using MTT cell proliferation assays.

### SUMMARY OF THE INVENTION

In one aspect, the invention provides an anti-IFNAR2 monoclonal antibody which blocks the binding of a first type I interferon to IFNAR2 and does not block the binding of a second type I interferon to IFNAR2.

In another aspect, the invention provides an anti-IFNAR2 monoclonal antibody that competes with an antibody selected from the group consisting of anti-IFNAR2 monoclonal antibodies 1F3, 3B7 and 1D3 for binding to IFNAR2.

In an additional aspect, the invention provides an anti-IFNAR2 monoclonal antibody selected from the group consisting of: (1) an antibody that binds to one or more of amino acid positions 49, 51, 52 and 54 in situ in IFNAR2; (2) an antibody that binds to one or more of amino acid positions 68, 71 and 72 in situ in IFNAR2; (3) an antibody that binds to one or more of amino acid positions 133, 134, 135 and 139 in situ in IFNAR2; (4) an antibody that binds to one or more of amino acid positions 153, 154 and 156 in situ in IFNAR2 ; (5) an antibody that binds to one or more of amino acid positions 74, 77 and 78 in situ in IFNAR2; and (6) an antibody that binds to one or more of amino acid positions 105 and 109 in situ in IFNAR2.

### BRIEF DESCRIPTION OF THE DRAWINGS

FIG. 1 is a graph depicting the ability of anti-IFNAR2 mAbs to inhibit the binding of IFN-α 2/1 to IFNAR2-IgG in an ELISA assay. IFNAR2-IgG was captured onto ELISA wells precoated with goat anti-human IgG Fc. Various concentrations of mabs and a predetermined suboptimal concentration of biotinylated IFN-α 2/1 (Bio-IFN-α 2/1) were added. The bound Bio-IFN-α 2/1 was detected by the addition of HRP-streptavidin.

FIG. 2 is an autoradiograph depicting the effect of anti-IFNAR2 mAbs on the IGSF3 formation induced by type I IFN. Hela cells were first incubated with mAbs followed by the addition of IFN-α8 at a concentration of 20 ng/ml. Twenty minutes later, cell lysates were prepared and IGSF complex was detected by electrophoretic mobility shift assay. Anti-IFN-αmAb 9E1 was included as a positive control.

FIGS. 3A and 3B are graphs depicting detailed IFNAR2 binding analyses of IFN-α2/1 and blocking mAb 1 F3, respectively. 0.5 nM concentrations of mutant IFNAR2-IgGs were captured onto ELISA wells precoated with goat anti-human IgG Fc. Various concentrations of Bio-IFN-α2/1 or mAb IF3 were added. The bound Bio-IFNα 2/1 and mAb IF3 were detected with HRP-streptavidin and HRP-goat anti-mouse IgG, respectively.

FIG. 4 is a model of hIFNAR2 displaying its protein sequence on the structural backbone of tissue factor. The model shows the location of some residues (in red) important for binding of mAbs to IFNAR2.

FIG. 5 depicts the DNA sequence (SEQ ID NO. 25) and amino acid sequence (SEQ ID NO. 26) of the IFNAR2 ECD-IgG coding insert in pRK5 hIFNAR2-IgG. The DNA sequence encoding the leader peptide amino acid sequence (corresponding to amino acids 1-26 in FIG. 1 on page 21608 of Domanski et al., J. Biol. Chem., 270: 21606-21611 (1995)) of IFNAR2 is shown as bases 22-99 of SEQ ID NO. 25 in FIG. 5. The leader peptide amino acid sequence is omitted from FIG. 5 in order to present the mature IFNAR2 ECD sequence as amino acids 1-216 of the IFNAR2 ECD-IgG fusion protein sequence (SEQ ID NO. 26). Unless otherwise indicated, the amino acid numbering scheme for IFNAR2 ECD shown in FIG. 5 is used throughout the application.

### METHODS OF CARRYING OUT THE INVENTION

A. Definitions

As used herein, the terms “type I interferon” and “human type I interferon” are defined as all species of native human interferon which fall within the human interferon-α, interferon-ω and interferonβ classes and which bind to a common cellular receptor. Natural human interferon-α comprises 23 or more closely related proteins encoded by distinct genes with a high degree of structural homology (Weissmann and Weber, Prog. Nucl Acid. Res. Mol Biol., 33: 251 (1986); J. Interferon Res., 13: 443-444 (1993)). The human IFN-α locus comprises two subfamilies. The first subfamily consists of at least 14 functional, non-allelic genes, including genes encoding IFN-αA (IFN-α2), IFN-αB (IFN-α8), IFN-αC (IFN-α10), IFN-αD (IFN-α1), IFN-αE (IFN-α22), IFN-αF (IFN-α21), IFN-αG (IFN-α5), and IFN-αH (IFN-α14), and pseudogenes having at least 80% homology. The second subfamily, α_{II} or ω, contains at least 5 pseudogenes and 1 flnctional gene (denoted herein as “IFN-α_{II}1” or “IFN-ω”) which exhibits 70% homology with the IFN-α genes (Weissmann and Weber (1986)). The human IFN-β is encoded by a single copy gene.

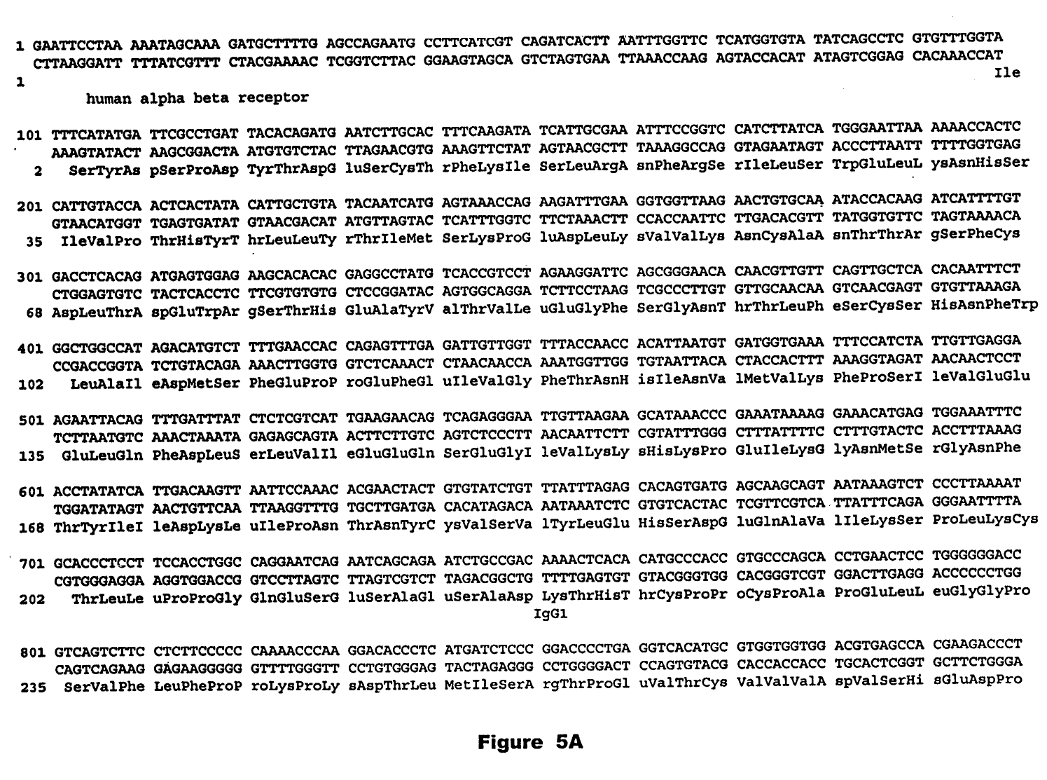

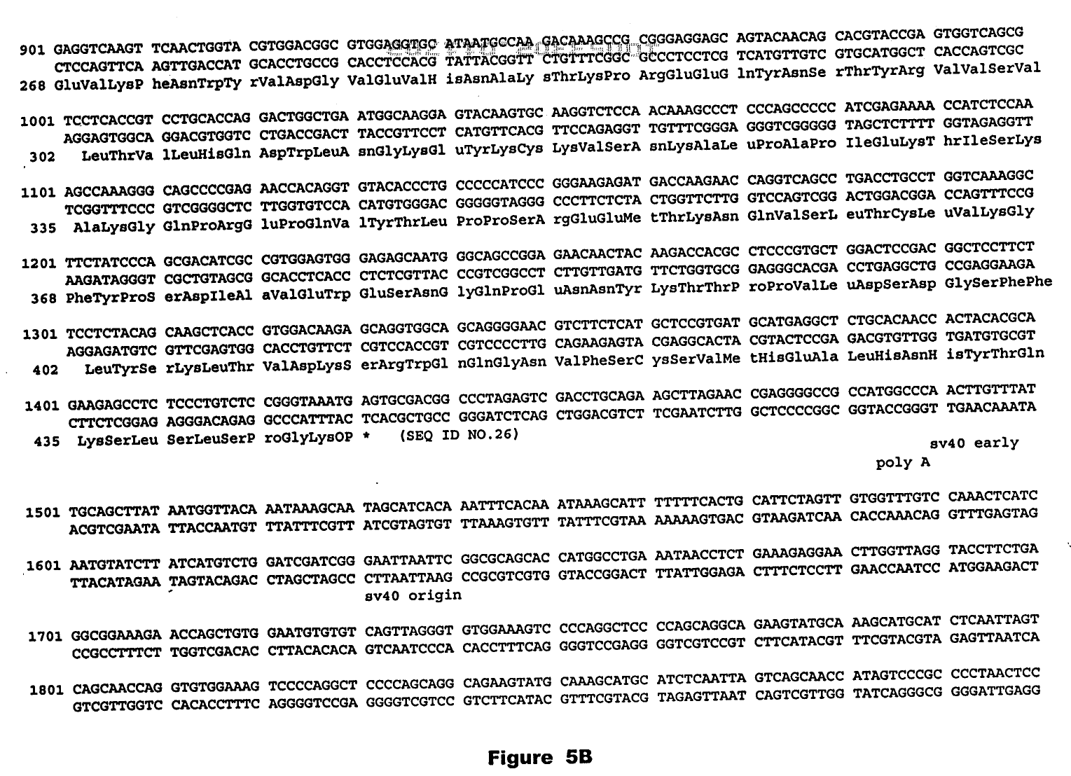

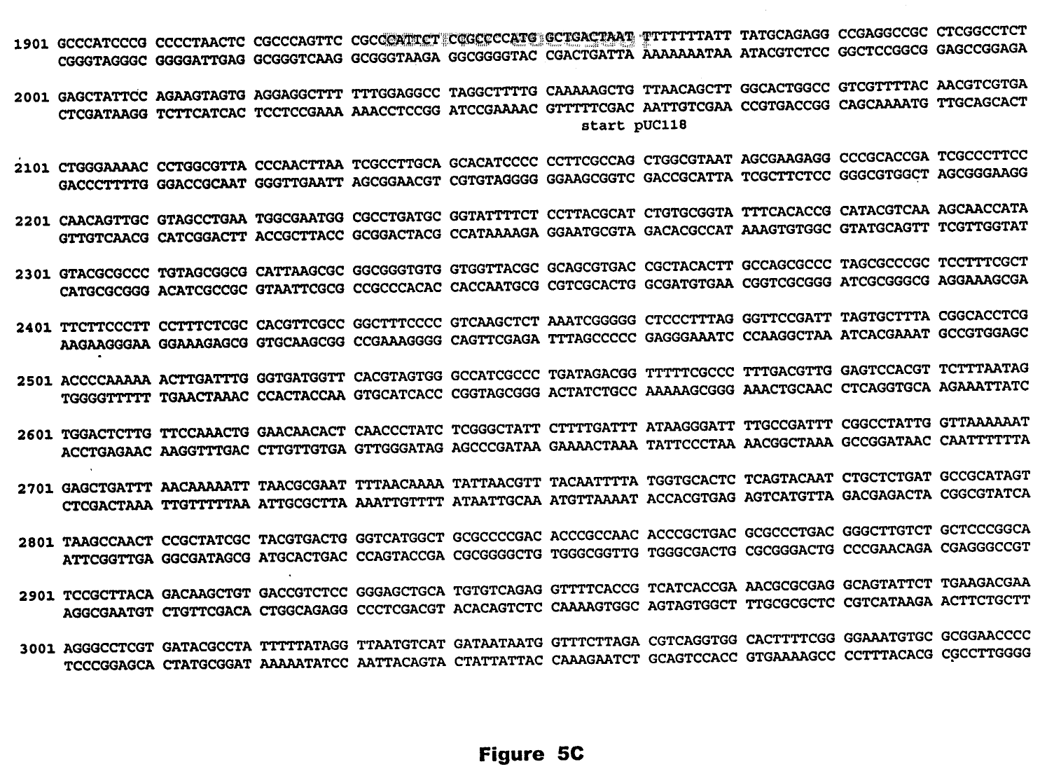

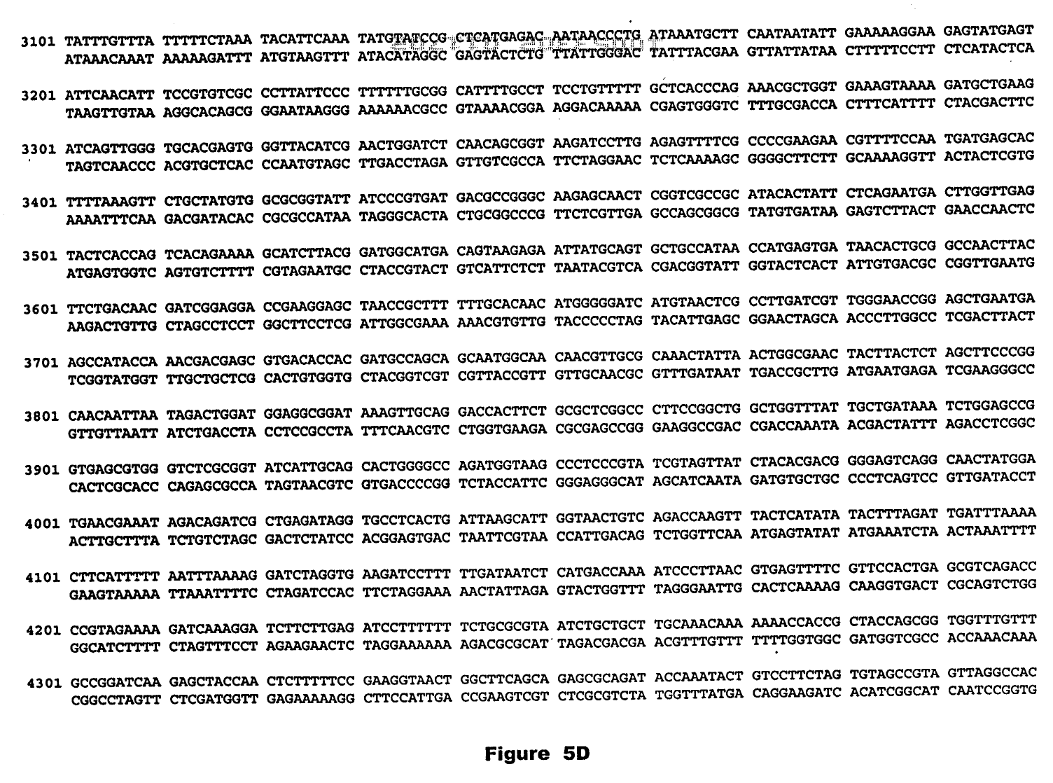

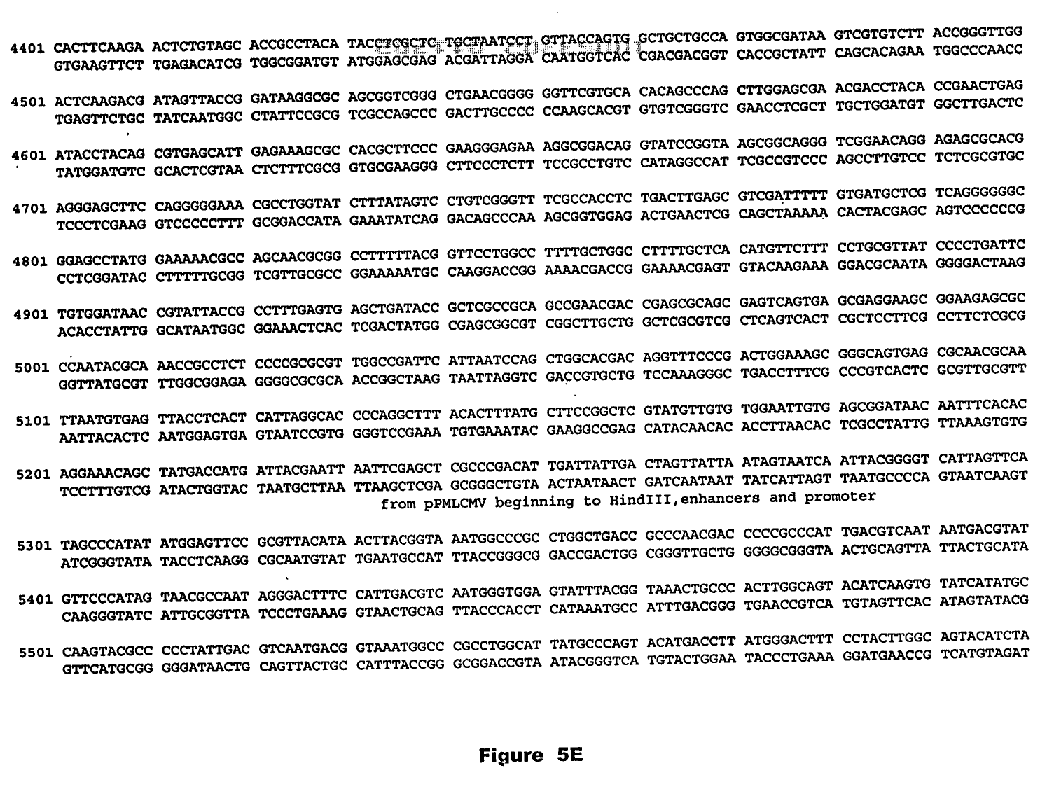

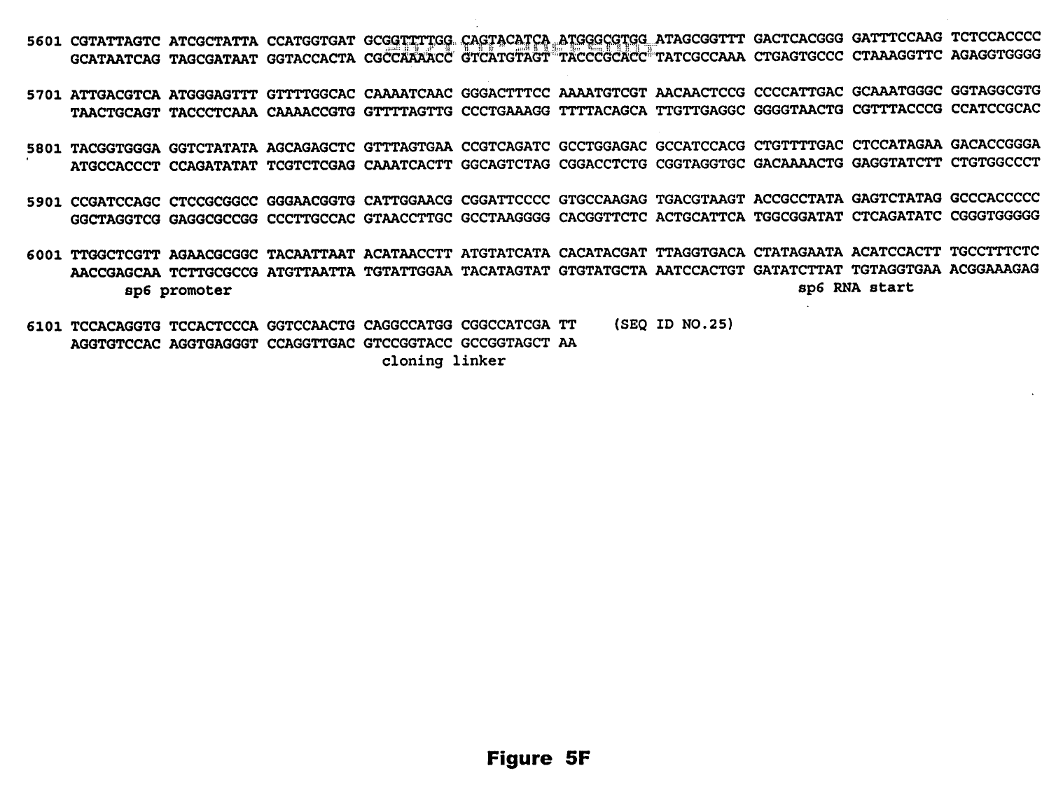

As used herein, the terms “first human interferon-α (hIFN-α) receptor”, “IFN-αR”, “hIFNAR1”, “IFNAR1”, and “Uze chain” are defined as the 557 amino acid receptor protein cloned by Uze et al., Cell, 60: 225-234 (1990), including an extracellular domain of 409 residues, a transmembrane domain of 21 residues, and an intracellular domain of 100 residues, as shown in FIG. 5 on page 229 of Uze et al. Also encompassed by the foregoing terms are fragments of IFNAR1 that contain the extracellular domain (ECD) (or fragments of the ECD) of IFNAR1.

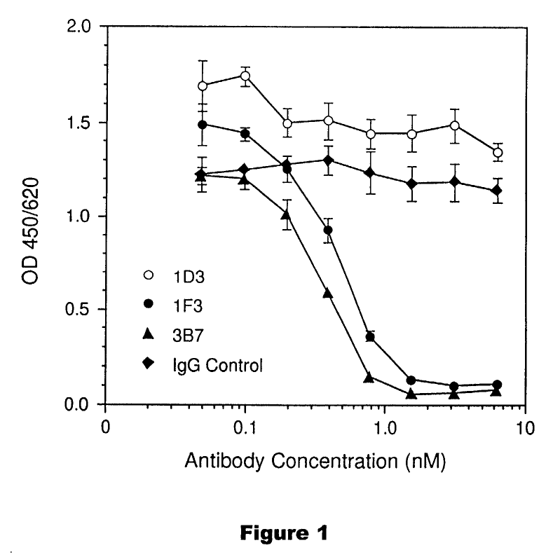

As used herein, the terms “second human interferon-α (hIFN-α) receptor”, “IFN-αR”, “hIFNAR2”, “IFNAR2”, and “Novick chain” are defined as the 515 amino acid receptor protein cloned by Domanski et al., J. Biol. Chem., 37: 21606-21611 (1995), including an extracellular domain of 217 residues, a transmembrane domain of 21 residues, and an intracellular domain of 250 residues, as shown in FIG. 1 on page 21608 of Domanski et al. Also encompassed by the foregoing terms are fragments of IFNAR2 that contain the extracellular domain (ECD) (or fragments of the ECD) of IFNAR2, and soluble forms of IFNAR2, such as IFNAR2 ECD fused to an immunoglobulin sequence, e.g. IFNAR2 ECD-IgG as described in the Example below.

As used herein, the term “anti-IFNAR2 antibody” is defined as an antibody that is capable of binding to IFNAR2.

As used herein, an anti-IFNAR2 antibody with the property or capability of “blocking the binding of a type I interferon to IFNAR2” is defined as an anti-IFNAR2 antibody capable of binding to IFNAR2 such that the ability of IFNAR2 to bind to one or more type I interferons is impaired or eliminated. An anti-IFNAR2 antibody candidate can be tested for such activity, for example, by adsorbing anti-IFNAR2 antibody to immobilized IFNAR2 followed by subjecting the adsorbed antibody to elution with an excess of a selected type I interferon. If an eluent comprising an excess of the selected type I interferon produces an eluate containing a greater concentration of the candidate antibody than the concentration of candidate antibody present in an eluate produced by a “blank” eluent (the same eluent containing no type I interferon) in a control elution, as determined by, e.g., radioimmunoassays performed on the respective eluates with radiolabelled, soluble IFNAR2, then the candidate antibody competes with the selected type I interferon for binding to IFNAR2. In some embodiments, the anti-IFNAR2 antibody of the invention competes with a selected type I interferon for binding to IFNAR2 and accordingly impairs or eliminates the binding of the selected type I interferon to IFNAR2.

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Press, NY, 1989). As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences ofProteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known.

The term “antibody” specifically covers monoclonal antibodies, including antibody fragment clones.

“Antibody fragments” comprise a portion of an intact antibody, generally the antigen binding or variable region of the intact antibody. Examples of antibody fragments include Fab, Fab′, F(ab′)₂, and Fv fragments; diabodies; single-chain antibody molecules, including single-chain Fv (scFv) molecules; and multispecific antibodies formed from antibody fragments.

The term “monoclonal antibody” as used herein refers to an antibody (or antibody fragment) obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al, Nature, 256:495 (1975), or may be made by recombinant DNA methods (see, e.g., U.S. Pat. No. 4,816,567). The “monoclonal antibodies” also include clones of antigen-recognition and binding-site containing antibody fragments (Fv clones) isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567 to Cabilly et aL; Morrison et al., Proc. Natl. Acad. Sci. USA, 81:6851-6855 (1984)).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a Primatized™ antibody wherein the antigen-binding region of the antibody is derived from an antibody produced by immunizing macaque monkeys with the antigen of interest.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human.

As used herein, the terms “each member of the group consisting of” and “each of” are synonymous.

As used herein, the terms “any member of the group consisting of” and “any of” are synonymous.

B. General Methods

In general, the invention provides anti-IFNAR2 antibodies that are useful for treatment of immune-mediated disorders in which a partial or total blockade of type I interferon activity is desired. In one embodiment, the anti-IFNAR2 antibodies of the invention are used to treat autoimmune disorders, such as type I and type II diabetes, systemic lupus erythematosis (SLE), and rheumatoid arthritis. In another embodiment, the anti-IFNAR2 antibodies provided herein are used to treat graft rejection or graft versus host disease. The unique properties of the anti-IFNAR2 antibodies of the invention make them particularly useful for effecting target levels of immunosuppression in a patient. For patients requiring acute intervention, the anti-IFNAR2 antibodies provided herein which cause broad spectrum ablation of type I interferon activity can be used to effect the largest possible compromise of an undesired immune response. For patients requiring maintenance immunosuppression, the anti-IFNAR2 antibodies provided herein which block one or more (but not all) species of type I interferon can be used to effect partial compromise of the patient's immune system in order to reduce the risk of undesirable immune responses while leaving some components of the patient's type I interferon-mediated immunity intact in order to avoid infection.

In another aspect, the anti-IFNAR2 antibodies of the invention find utility as reagents for detection and isolation of IFNAR2, such as detection of IFNAR2 expression in various cell types and tissues, including the determination of IFNAR2 receptor density and distribution in cell populations, and cell sorting based on IFNAR2 expression. In yet another aspect, the present anti-IFNAR2 antibodies are useful for the development of IFNAR2 antagonists with type I interferon blocking activity patterns similar to those of the subject antibodies. The anti-IFNAR2 antibodies of the invention can be used in IFNAR2 signal transduction assays to screen for small molecule antagonists of IFNAR2 which will exhibit similar pharmacological effects in blocking the binding of type I interferons to IFNAR2.

I. Methods of Making Synthetic Anti-IFNAR2 Fv Clones

The anti-IFNAR2 antibodies of the invention can be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-IFNAR2 antibodies of the invention can be obtained by designing a suitable antigen screening procedure to select for the phage clone of interest followed by construction of a full length anti4FNAR2 antibody clone using the Fv sequences from the phage clone of interest and suitable constant region (Fc) sequences described in Kabat et al., Sequences of proteins of Immunological Interest, Fifth Edition, NIH Publication 91-3242, Bethesda Md. (1991), vols. 1-3.

1. Construction of Phage Libraries

The antigen-binding domain of an antibody is formed from two variable (V) regions of about 110 amino acids, one each from the light (VL) and heavy (VH) chains, that both present three hypervariable loops or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994). As used herein, scFv encoding phage clones and Fab encoding phage clones are collectively referred to as “Fv phage clones” or “Fv clones”.

The naive repertoire of an animal (the repertoire before antigen challenge) provides it with antibodies that can bind with moderate affinity (K_{d}⁻¹ of about 10⁶ to 10⁷ M⁻¹) to essentially any non-self molecule. The sequence diversity of antibody binding sites is not encoded directly in the germline but is assembled in a combinatorial manner from V gene segments. In human heavy chains, the first two hypervariable loops (H1 and H2) are drawn from less than 50 VH gene segments, which are combined with D segments and JH segments to create the third hypervariable loop (H3). In human light chains, the first two hypervariable loops (L1 and L2) and much of the third (L3) are drawn from less than approximately 30 Vλ and less than approximately 30 Vκ segments to complete the third hypervariable loop (L3).

Each combinatorial rearrangement of V-gene segments in stem cells gives rise to a B cell that expresses a single VH-VL combination. Immunizations triggers any B cells making a combination that binds the immunogen to proliferate (clonal expansion) and to secrete the corresponding antibody. These naive antibodies are then matured to high affinity (K_{d}⁻¹≧10⁹ M⁻¹) by a process of mutagenesis and selection known as affinity maturation. It is after this point that cells are normally removed to prepare hybridomas and generate high-affinity monoclonal antibodies.

At three stages of this process, repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

Phage display mimics the B cell. Filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g. as described by Marks et al., J. Mol. Biol., 222: 581-597 (1991), or as Fab fragments, in which one chain is fused to plll and the other is secreted into the bacterial host cell periplasm where assembly of a Fab-coat protein structure which becomes displayed on the phage surface by displacing some of the wild type coat proteins, e.g. as described in Hoogenboom et al., Nucl. Acids Res., 19: 4133-4137 (1991). When antibody fragments are fused to the N-terminus of pIII, the phage is infective. However, if the N-terminal domain of pIII is excised and fusions made to the second domain, the phage is not infective, and wild type pIII must be provided by helper phage. The pIII fusion and other proteins of the phage can be encoded entirely within the same phage replicon, or on different replicons. When two replicons are used, the pIII fusion is encoded on a phagemid, a plasmid containing a phage origin of replication. Phagemids can be packaged into phage particles by “rescue” with a helper phage such as M13K07 that provides all the phage proteins, including pIII, but due to a defective origin is itself poorly packaged in competitions with the phagemids as described in Vieira and Messing, Meth. Enzymol., 153: 3-11 (1987). In a preferred method, the phage display system is designed such that the recombinant phage can be grown in host cells under conditions permitting no more than a minor amount of phage particles to display more than one copy of the Fv-coat protein fusion on the surface of the particle as described in Bass et al., Proteins, 8: 309-314 (1990) and in WO 92/09690 (PCT/US91/09133 published June 11, 1992).

In general, nucleic acids encoding antibody gene fragments are obtained from immune cells harvested from humans or animals. If a library biased in favor of anti-IFNAR2 clones is desired, the subject is immunized with IFNAR2 to generate an antibody response, and spleen cells and/or circulating B cells other peripheral blood lymphocytes (PBLs) are recovered for library construction. In a preferred embodiment, a human antibody gene fragment library biased in favor of anti-human IFNAR2 clones is obtained by generating an anti-human IFNAR2 antibody response in transgenic mice carrying a functional human immunoglobulin gene array (and lacking a functional endogenous antibody production system) such that IFNAR2 immunization gives rise to B cells producing human antibodies against IFNAR2. The generation of human antibody-producing transgenic mice is described in Section B(III)(b) below.

Additional enrichment for anti-IFNAR2 reactive cell populations can be obtained by using a suitable screening procedure to isolate B cells expressing IFNAR2-specific membrane bound antibody, e.g., by cell separation with IFNAR2 affinity chromatography or adsorption of cells to fluorochrome-labelled IFNAR2 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which IFNAR2 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86: 3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-546 (1989). However, for amplifying from cDNA, back primers can also be based in the leader exon as described in Jones et al., Biotechnol., 9: 88-89 (1991), and forward primers within the constant region as described in Sastry et al., Proc. Natl. Acad. Sci. (USA), 86: 5728-5732 (1989). To maximize complementarity, degeneracy can be incorporated in the primers as described in Orlandi et al. (1989) or Sastry et al. (1989). Preferably, the library diversity is maximized by using PCR primers targeted to each V-gene family in order to amplify all available VH and VL arrangements present in the immune cell nucleic acid sample, e.g. as described in the method of Marks et al., J. Mol Biol., 222: 581-597 (1991) or as described in the method of Orum et al., Nucleic Acids Res., 21: 4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human VH-gene segments have been cloned and sequenced (reported in Tomlinson et al., J. Mol Biol., 227: 776-798 (1992)), and mapped (reported in Matsuda et al., Nature Genet., 3: 88-94 (1993); these cloned segments (including all the major comformations of the H1 and H2 loop) can be used to generate diverse VH gene repertoires with PCR primers encoding H3 loops of diverse sequence and length as described in Hoogenboom and Winter, J. Mol. Biol., 227: 381-388 (1992). VH repertoires can also be made with all the sequence diversity focussed in a long H3 loop of a single length as described in Barbas et al., Proc. Natl. Acad. Sci. USA, 89: 4457-4461 (1992). Human Vκ and Vλ segments have been cloned and sequenced (reported in Williams and Winter, Eur. J. Immunol., 23: 1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128: 119-126 (1993), or in vivo by combinatorial infection, e.g., the loxP system described in Waterhouse et al., Nucl. Acids Res., 21: 2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions. These huge libraries provide large numbers of diverse antibodies of good affinity (K_{d}⁻¹ of about 10⁻⁸ M).

Alternatively, the repertoires may be cloned sequentially into the same vector, e.g. as described in Barbas et al., Proc. Natl. Acad. Sci. USA, 88: 7978-7982 (1991), or assembled together by PCR and then cloned, e.g. as described in Clackson et al., Nature, 352: 624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al, Nucl. Acids Res., 20: 3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1: 11-15 (1989)) in the method of Hawkins et al., J. Mol. Biol., 226: 889-896 (1992) or in the method of Gram et al., Proc. Natl. Acad. Sci USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published Mar. 14, 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with affinities in the 10⁻⁹ M range.

2. Panning Phage Display Libraries for Anti-IFNAR2 Clones

a. Synthesis of IFNAR2 and IFNAR2 Ligands

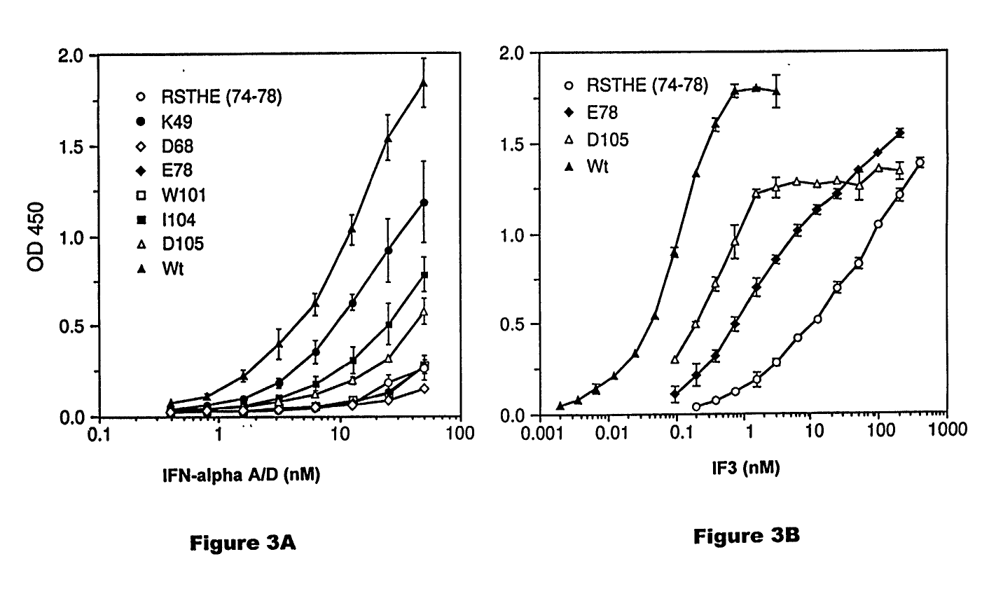

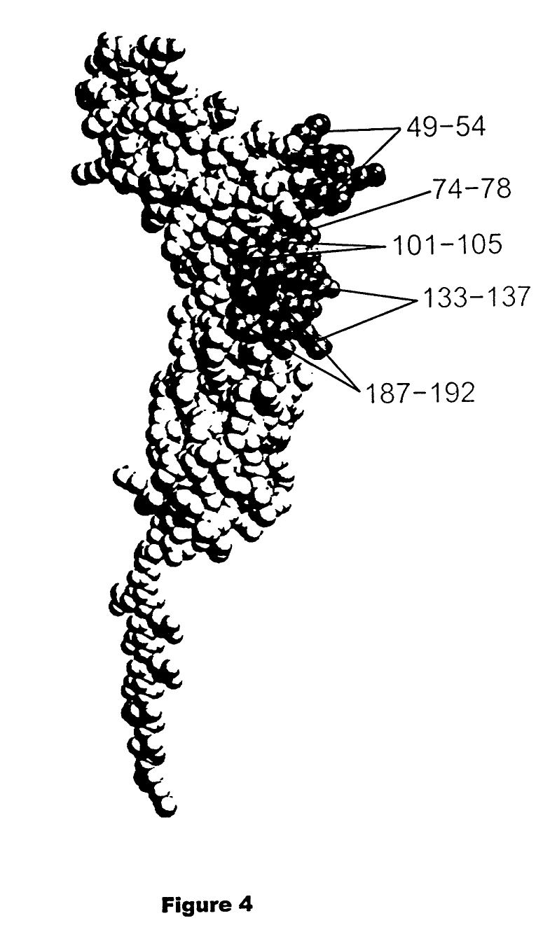

Nucleic acid sequence encoding the IFNAR2s used herein can be designed using the amino acid sequence of the desired region of IFNAR2, e.g. the extracellular domain spanning amino acids 27 to 243 of FIG. 7 on page 395 of Novick et al., Cell, 77: 391-400 (1994). Alternatively, the cDNA sequence of FIG. 7 on page 395 of Novick et al., supra, can be used. In addition, nucleic acid encoding an immunoglobulin G (IgG)-IFNAR2 extracellular domain fusion protein can be obtained from the amino acid or cDNA sequence shown in FIG. 5 below. Likewise, nucleic acid sequence encoding the human type I interferons used herein can be designed using published amino acid and nucleic acid sequences, e.g. see the J. Interferon Res., 13: 443-444 (1993) compilation of references containing genomic and cDNA sequences for various type I interferons, and the references cited therein. For the IFN-αA, IFN-αB, IFN-αC, IFN-αD, IFN-αE, IFN-αF, IFN-αG, and IFN-αH amino acid sequences or cDNA sequences, see FIGS. 3 and 4 on pages 23-24 of Goeddel et al., Nature, 290: 20-26 (1981). For cDNA encoding the amino acid sequence of IFN-α_{II}1 (IFN-ω), see Capon et al., Mol. Cell. Biol., 5: 768-779 (1985) and Hauptmann and Swetly, Nucleic Acids Res., 13: 4739-4749 (1985). For cDNA encoding the amino acid sequence of IFN-β, see Taniguchi et al., Proc. Jpn. Acad. Ser. B, 55: 464-469 (1979); Taniguchi et al., Gene, 10: 11-15 (1980); and U.S. Pat. No. 5,460,811 to Goeddel and Crea. For cDNA encoding the amino acid sequence of IFN-α7, see Cohen et al., Dev. Biol.Standard, 60: 111-122 (1985). DNAs encoding the IFNAR2s or type I interferons of interest can be prepared by a variety of methods known in the art. These methods include, but are not limited to, chemical synthesis by any of the methods described in Engels et al., Agnew. Chem. Int. Ed. Engl., 28: 716-734 (1989), such as the triester, phosphite, phosphoramidite and H-phosphonate methods. In one embodiment, codons preferred by the expression host cell are used in the design of the IFNAR2 or type I interferon-encoding DNA. Alternatively, DNA encoding the IFNAR2 or type I interferon can be isolated from a genomic or cDNA library.

For production of the mutant IFNAR2s used herein, DNA sequence encoding wild type IFNAR2 can be altered to encode the desired IFNAR2 mutant by using recombinant DNA techniques, such as site specific mutagenesis (Kunkel et al., Methods Enzymol. 204:125-139 (1991); Carter, P., et al., Nucl. Acids. Res. 13:4331 (1986); Zoller, M. J. et al.,Nucl. Acids Res. 10:6487 (1982)), cassette mutagenesis (Wells, J. A., et al.,Gene 34:315 (1985)), restriction selection mutagenesis (Wells, J. A., et al., Philos. Trans, R. Soc. London SerA 317: 415 (1986)), and the like.

Following construction of the DNA molecule encoding the IFNAR2 or type I interferon of interest, the DNA molecule is operably linked to an expression control sequence in an expression vector, such as a plasmid, wherein the control sequence is recognized by a host cell transformed with the vector. In general, plasmid vectors contain replication and control sequences which are derived from species compatible with the host cell. The vector ordinarily carries a replication site, as well as sequences which encode proteins that are capable of providing phenotypic selection in transformed cells.

For expression in prokaryotic hosts, suitable vectors include pBR322 (ATCC No. 37,017), phGH107 (ATCC No. 40,011), pBO475, pS0132, pRIT5, any vector in the pRIT20 or pRIT30 series (Nilsson and Abralunsen, Meth. Enzymol., 185: 144-161 (1990)), pRIT2T, pKK233-2, pDR540 and pPL-lambda. Prokaryotic host cells containing the expression vectors suitable for use herein include E. coli K12 strain 294 (ATCC NO. 31446), E. coli strain JM101 (Messing et al., Nucl. Acid Res., 9: 309 (1981)), E. coli strain B, E. coli strain χ1776 (ATCC No. 31537), E. coli c600 (Appleyard, Genetics, 39: 440 (1954)), E. coli W3110 (F-, gamma-, prototrophic, ATCC No. 27325), E. coli strain 27C7 (W3110, tonA, phoA E15, (argF-lac)169, ptr3, degP41, ompT, kan^{r}) (U.S. Pat. No. 5,288,931, ATCC No. 55,244), Bacillus subtilis, Salmonella typhimurium, Serratia marcesans, and Pseudomonas species.

In addition to prokaryotes, eukaryotic organisms, such as yeasts, or cells derived from multicellular organisms can be used as host cells. For expression in yeast host cells, such as common baker's yeast or Saccharomyces cerevisiae, suitable vectors include episomally replicating vectors based on the 2-micron plasmid, integration vectors, and yeast artificial chromosome (YAC) vectors. For expression in insect host cells, such as Sf9 cells, suitable vectors include baculoviral vectors. For expression in plant host cells, particularly dicotyledonous plant hosts, such as tobacco, suitable expression vectors include vectors derived from the Ti plasmid of Agrobacterium tumefaciens.

However, interest has been greatest in vertebrate host cells. Examples of useful mammalian host cells include monkey kidney CVI line transformed by SV40 (COS-7, ATCC CRL 1651); human embryonic kidney line (293 or 293 cells subcloned for growth in suspension culture, Graham et al., J. Gen Virol., 36: 59 (1977)); baby hamster kidney cells (BHK, ATCC CCL 10); Chinese hamster ovary cells/-DHFR (CHO, Urlaub and Chasin, Proc. Natl. Acad. Sci. USA, 77: 4216 (1980)); mouse sertoli cells (TM4, Mather, Biol. Reprod., 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383: 44-68 (1982)); MRC5 cells; FS4 cells; and a human hepatoma cell line (Hep G2). For expression in mammalian host cells, useful vectors include vectors derived from SV40, vectors derived from cytomegalovirus such as the pRK vectors, including pRK5 and pRK7 (Suva et al., Science, 237: 893-896 (1987), EP 307,247 (3/15/89), EP 278,776 (8/17/88)) vectors derived from vaccinia viruses or other pox viruses, and retroviral vectors such as vectors derived from Moloney's murine leukemia virus (MoMLV).

Optionally, the DNA encoding the IFNAR2 or type I interferon of interest is operably linked to a secretory leader sequence resulting in secretion of the expression product by the host cell into the culture medium. Examples of secretory leader sequences include stil, ecotin, lamB, herpes GD, lpp, alkaline phosphatase, invertase, and alpha factor. Also suitable for use herein is the 36 amino acid leader sequence of protein A (Abrahmsen et al., EMBO J., 4: 3901(1985)).

Host cells are transfected and preferably transformed with the above-described expression or cloning vectors of this invention and cultured in conventional nutrient media modified as appropriate for inducing promoters, selecting transformnants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., Molecular Cloning (2nd ed.), Cold Spring Harbor Laboratory, NY (1989), is generally used for prokaryotes or other cells that contain substantial cell-wall barriers. Infection with Agrobacterium tumefaciens is used for transformation of certain plant cells, as described by Shaw et al., Gene, 23: 315 (1983) and WO 89/05859 published Jun. 29, 1989. For mammalian cells without such cell walls, the calcium phosphate precipitation method described in sections 16.30-16.37 of Sambrook et al., supra, is preferred. General aspects of mammalian cell host system transformations have been described by Axel in U.S. Pat. No. 4,399,216 issued Aug. 16, 1983. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells such as by nuclear injection, electroporation, or by protoplast fusion may also be used.

Prokaryotic host cells used to produce the IFNAR2 or type I interferon of interest can be cultured as described generally in Sambrook et al., supra.

The mammalian host cells used to produce the IFNAR2 or type I interferon of interest can be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham and Wallace, Meth. Enz., 58: 44 (1979), Barnes and Sato, Anal. Biochem., 102: 255 (1980), U.S. Pat. Nos. 4,767,704; 4,657,866; 4,927,762; or 4,560,655; WO 90/03430; WO 87/00195; U.S. Pat. Re. No. 30,985; or U.S. Pat. No. 5,122,469, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in in vitro culture as well as cells that are within a host animal.

In an intracellular expression system or periplasmic space secretion system, the recombinantly expressed IFNAR2 or type I interferon protein can be recovered from the culture cells by disrupting the host cell membrane/cell wall (e.g. by osmotic shock or solubilizing the host cell membrane in detergent). Alternatively, in an extracellular secretion system, the recombinant protein can be recovered from the culture medium. As a first step, the culture medium or lysate is centrifuged to remove any particulate cell debris. The membrane and soluble protein fractions are then separated. Usually, the IFNAR2 or type I interferon is purified from the soluble protein fraction. If the IFNAR2 is expressed as a membrane bound species, the membrane bound peptide can be recovered from the membrane fraction by solubilization with detergents. The crude peptide extract can then be further purified by suitable procedures such as fractionation on irnmunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins and ligand affinity using IFNAR2 (for type I interferon purification) or type I interferons or anti-IFNAR2 antibodies (for IFNAR2 purification) immobilized on a matrix.

Many of the human type I interferons used herein can be obtained from commercial sources, e.g. human IFN-β is available from Sigma (St. Louis, Mo.).

b. Immobilization of IFNAR2

The purified IFNAR2 can be attached to a suitable matrix such as agarose beads, acrylamide beads, glass beads, cellulose, various acrylic copolymers, hydroxyl methacrylate gels, polyacrylic and polymethacrylic copolymers, nylon, neutral and ionic carriers, and the like, for use in the affinity chromatographic separation of phage display clones. Attachment of the IFNAR2 protein to the matrix can be accomplished by the methods described in Methods in Enzymology, vol. 44 (1976). A commonly employed technique for attaching protein ligands to polysaccharide matrices, e.g. agarose, dextran or cellulose, involves activation of the carrier with cyanogen halides and subsequent coupling of the peptide ligand's primary aliphatic or aromatic amines to the activated matrix.

Alternatively, IFNAR2 can be used to coat the wells of adsorption plates, expressed on host cells affixed to adsorption plates or used in cell sorting, or conjugated to biotin for capture with streptavidin-coated beads, or used in any other art-known method for panning phage display libraries.

c. Panning Procedures

The phage library samples are contacted with immobilized IFNAR2 under conditions suitable for binding of at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci USA, 88: 7978-7982 (1991), or by alkali, e.g. as described in Marks et al., J. Mol. Biol., 222: 581-597 (1991), or by IFNAR2 antigen or type I interferon ligand competition, e.g. in a procedure similar to the antigen competition method of Clackson et al., Nature, 352: 624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for IFNAR2. However, random mutation of a selected antibody (e.g. as performed in some of the affinity maturation techniques described above) is likely to give rise to many mutants, most binding to antigen, and a few with higher affinity. With limiting IFNAR2, rare high affinity phage could be competed out. To retain all the higher affinity mutants, phages can be incubated with excess biotinylated IFNAR2, but with the biotinylated IFNAR2 at a concentration of lower molarity than the target molar affinity constant for IFNAR2. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

3. Activity Selection of Anti-IFNAR2 Clones

In one embodiment, the invention provides anti-IFNAR2 antibodies that block the binding between a first type I interferon and IFNAR2 and do not block the binding between a second type I interferon and IFNAR2. Fv clones corresponding to such anti-IFNAR2 antibodies can be selected by (1) isolating anti-IFNAR2 clones from a phage library as described in Section B(I)(2) above, and optionally amplifying the isolated population of phage clones by growing up the population in a suitable bacterial host; (2) selecting a first type I interferon and a second type I interferon against which blocking and non-blocking activity, respectively, is desired; (3) adsorbing the anti-IFNAR2 phage clones to immobilized IFNAR2; (4) using an excess of the first type I interferon to elute the adsorbed clones that recognize IFNAR2-binding determinants which overlap or are shared with the IFNAR2-binding determinants of the first type I interferon; (5) readsorbing the clones isolated from step (4) to immobilized IFNAR2; (6) using an excess of the second type I interferon to elute any undesired clones that recognize IFNAR2-binding determinants which overlap or are shared with the IFNAR2-binding determinants of the second type I interferon; and (7) eluting the clones which remain adsorbed following step (6). The IFNAR2-binding competitions used in this process allow the efficient selection of a phage clone that can block the binding of one selected type I interferon to IFNAR2 and that cannot block the binding of a second selected type I interferon to IFNAR2. Optionally, clones with the desired blocking/non-blocking properties can be further enriched by repeating the selection procedures described herein one or more times.

In another embodiment, the anti-IFNAR2 Fv clone is selected by using a second type I interferon that is IFN-αD in steps (1)-(7) above.

Also provided herein are anti-IFNAR2 Fv clones selected by using a second type I interferon that is IFN-αA in steps (1)-(7) above.

In yet another embodiment, the anti-IFNAR2 Fv clone is selected by using a second type I interferon that is IFN-αB in steps (1)-(7) above.

Additionally provided herein are anti-IFNAR2 Fv clones selected by using a second type I interferon that is IFN-α_{II}1 in steps (1)-(7) above.

Further provided herein are anti-IFNAR2 Fv clones selected by using a second type I interferon that is IFN-β in steps (1)-(7) above.

Also encompassed herein are anti-IFNAR2 Fv clones selected by a first type I interferon that is selected from the group consisting of IFN-αD, IFN-αA, IFN-αG and IFN-αB and using a second type I interferon that is IFN-β in steps (1)-(7) above.

The invention additionally provides anti-IFNAR2 antibodies and Fv clones which block the binding of a more than one selected type I interferon to IFNAR2 and which do not block the binding of another type I interferon to IFNAR2. These Fv clones can be selected by repeating steps (4) and (5) in the above procedure for each type I interferon against which blocking activity is desired, i.e., after eluting clones with an excess of one of the type I interferons against which blocking activity is desired, the eluted clones can be readsorbed to the immobilized IFNAR2 and then subjected to an excess of another type I interferon against which blocking activity is desired, and the process can be repeated until the remaining clones have been eluted from immobilized IFNAR2 by each species of type I interferon against which blocking activity is desired. In one embodiment, an anti-IFNAR2 Fv clone that blocks the binding of IFN-αD, IFN-αA, IFN-αG and IFN-αB to IFNAR2 and does not block the binding of IFN-β to IFNAR2 is selected by eluting clones adsorbed to immobilized IFNAR2 with each of IFN-αD, IFN-αA, IFN-αG and IFN-αB in consecutive repetitions of steps (4) and (5) followed by subjecting IFNAR2-adsorbed clones to elution with IFN-β in step (6) of the above procedure.

The invention further encompasses anti-IFNAR2 antibodies which bind to specific determinant(s) on IFNAR2. Fv clones corresponding to such anti-IFNAR2 antibodies can be conveniently selected by adsorbing library clones to immobilized IFNAR2 mutants containing Ala substitutions at the specific determinants of interest and recovering library clones which fail to adsorb to immobilized, mutant IFNAR2 (i.e. collected from the column flow-through fractions). Next, the recovered clones are adsorbed to immobilized, wild type IFNAR2, and then the adsorbed clones are recovered, e.g. by elution with excess wild type IFNAR2. The first adsorption step removes clones that bind to IFNAR2 but do not bind to the selected determinant(s), and the second adsorption step removes clones that do not bind to IFNAR2 at all, leaving a population of clones enriched for binding to the selected IFNAR2 determinant(s). The desired clone will exhibit binding activity with wild type IFNAR2 that is greater than the clone's binding activity with the corresponding Ala-substituted IFNAR2 mutant (i.e. a binding level with wild type IFNAR2 that is above the background binding level with mutant IFNAR2). Optionally, the desired clone will exhibit binding activity with the corresponding Ala-substituted IFNAR2 mutant that is less than at or about 90%, or less than at or about 70%, or less than at or about 50%, or less than at or about 30%, or less than at or about 20%, or less than at or about 10%, or at or about 0% of the clone's binding activity with wild type IFNAR2.

Optionally, clones that bind to selected IFNAR2 determinants can be further enriched by repeating the selection procedures described herein one or more times.

In one embodiment, the invention provides anti-IFNAR2 antibodies and Fv clones which bind to a determinant that contains one or more of amino acids 49, 51, 52 and 54 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing Ala substitutions at amino acids 49, 51, 52 and 54 in order to adsorb undesired clones which bind to determinants on IFNAR2 that do not overlap with amino acid positions 49, 51, 52 or 54; (3) recovering the clones which fail to adsorb to immobilized, mutant IFNAR2 from the flow-through fractions in step (2); (4) readsorbing the recovered clones to immobilized, wild type IFNAR2; and (5) recovering the adsorbed clones by elution with excess IFNAR2.

In another embodiment, the invention provides anti-IFNAR2 antibodies and Fv clones which bind to a determinant that contains one or more of amino acids 68, 71 and 72 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing Ala substitutions at amino acids 68, 71 and 72 in order to adsorb undesired clones which bind to determinants on IFNAR2 that do not overlap with amino acid positions 68, 71 or 72; (3) recovering the clones which fail to adsorb to immobilized, mutant IFNAR2 from the flow-through fractions in step (2); (4) readsorbing the recovered clones to immobilized, wild type IFNAR2; and (5) recovering the adsorbed clones by elution with excess IFNAR2.

In yet another embodiment, the invention provides anti-IFNAR2 antibodies and Fv clones which bind to a determinant that contains one or more of amino acids 74, 77 and 78 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing Ala substitutions at amino acids 74, 77 and 78 in order to adsorb undesired clones which bind to determinants on IFNAR2 that do not overlap with amino acid positions 74, 77 or 78; (3) recovering the clones which fail to adsorb to immobilized, mutant IFNAR2 from the flow-through fractions in step (2); (4) readsorbing the recovered clones to immobilized, wild type IFNAR2; and (5) recovering the adsorbed clones by elution with excess IFNAR2.

In still another embodiment, the invention provides anti-IFNAR2 antibodies and Fv clones which bind to a determinant that contains one or both of amino acids 105 and 109 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing Ala substitutions at amino acids 105 and 109 in order to adsorb undesired clones which bind to determinants on IFNAR2 that do not overlap with amino acid positions 105 or 109; (3) recovering the clones which fail to adsorb to immobilized, mutant IFNAR2 from the flow-through fractions in step (2); (4) readsorbing the recovered clones to immobilized, wild type IFNAR2; and (5) recovering the adsorbed clones by elution with excess IFNAR2.

In a further embodiment, the invention provides anti-IFNAR2 antibodies and Fv clones which bind to a determinant that contains one or more of amino acids 133, 134, 135 and 139 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing Ala substitutions at amino acids 133, 134, 135 and 139 in order to adsorb undesired clones which bind to determinants on IFNAR2 that do not overlap with amino acid positions 133, 134, 135 or 139; (3) recovering the clones which fail to adsorb to immobilized, mutant IFNAR2 from the flow-through fractions in step (2); (4) readsorbing the recovered clones to immobilized, wild type IFNAR2; and (5) recovering the adsorbed clones by elution with excess IFNAR2.

In an additional embodiment, the invention provides anti-IFNAR2 antibodies and Fv clones which bind to a determinant that contains one or more of amino acids 153, 154 and 156 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing Ala substitutions at amino acids 153, 154 and 156 in order to adsorb undesired clones which bind to determinants on IFNAR2 that do not overlap with amino acid positions 153, 154 or 156; (3) recovering the clones which fail to adsorb to immobilized, mutant IFNAR2 from the flow-through fractions in step (2); (4) readsorbing the recovered clones to immobilized, wild type IFNAR2; and (5) recovering the adsorbed clones by elution with excess IFNAR2.

Also encompassed herein are anti-IFNAR2 antibodies and Fv clones which bind to a determinant that contains one or more of amino acids 49, 51, 52, and 54 in situ in the sequence of IFNAR2, and contains one or more of amino acids 68, 71, and 72 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing an Ala substitutions at amino acids 49, 51, 52, and 54 in order to adsorb undesired clones which bind to determinants on IFNAR2 that do not overlap with amino acid positions 49, 51, 52 or 54; (3) recovering the clones which fail to adsorb to immobilized, mutant IFNAR2 from the flow-through fractions in step (2); (4) repeating steps (2) and (3) using immobilized, mutant IFNAR2 containing Ala substitutions at amino acid positions 68, 71 and 72 as the adsorbent in order to remove undesired clones which bind to determinants on IFNAR2 that do not overlap with amino acid positions 68, 71 or 72; (5) readsorbing the recovered clones to immobilized, wild type IFNAR2; and (6) recovering the adsorbed clones by elution with excess IFNAR2.

Further provided herein are anti-IFNAR2 antibodies and Fv clones which bind to a determinant that contains one or more of amino acids 49, 51, 52, and 54 in situ in the sequence of IFNAR2, contains one or more of amino acids 68, 71, and 72 in situ in the sequence of IFNAR2, contains one or more of amino acids 74, 77, and 78 in situ in the sequence of IFNAR2, contains one or both of amino acids 105 and 109 in situ in the sequence of IFNAR2, contains one or more of amino acids 133, 134, 135, and 139 in situ in the sequence of IFNAR2, and contains one or more of amino acids 153, 154 and 156 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing an Ala substitutions at amino acids 49, 51, 52, and 54 in order to adsorb undesired clones which bind to determinants on IFNAR2 that do not overlap with amino acid positions 49, 51, 52 or 54; (3) recovering the clones which fail to adsorb to immobilized, mutant IFNAR2 from the flow-through fractions in step (2); (4) repeating steps (2) and (3) in order to screen the recovered clones for non-adsorption to the corresponding inmmobilized, Ala-substitution mutant IFNAR2 for each combination of amino acid positions that remains to be tested; (5) readsorbing the recovered clones to immobilized, wild type IFNAR2; and (6) recovering the adsorbed clones by elution with excess IFNAR2.

Additionally provided herein are anti-IFNAR2 antibodies and Fv clones which bind to a determinant that contains one or more of amino acids 133, 134, 135, and 139 in situ in the sequence of IFNAR2, and contains one or more of amino acids 153, 154 and 156 in situ in the sequence of IFNAR2. These Fv clones can be selected by (1) isolating anti-IFNAR2 clones from a phage library as described in Section B(I)(2) above, and optionally amplifying the isolated population of phage clones by growing up the population in a suitable bacterial host; (2) adsorbing anti-IFNAR2 phage clones to an immobilized, mutant IFNAR2 containing an Ala substitutions at amino acids 133, 134, 135, and 139 in order to adsorb undesired clones which bind to determinants on IFNAR2 that do not overlap with amino acid positions 133, 134, 135, or 139; (3) recovering the clones which fail to adsorb to immobilized, mutant IFNAR2 from the flow-through fractions in step (2); (4) repeating steps (2) and (3) in order to screen the recovered clones for non-adsorption to the corresponding immobilized, Ala-substitution mutant IFNAR2 for amino acid positions 153, 154 and 156; (5) readsorbing the recovered clones to immobilized, wild type IFNAR2; and (6) recovering the adsorbed clones by elution with excess IFNAR2.

In other embodiments, the invention provides anti-IFNAR2 antibodies and Fv clones which possess combinations of the differential type I interferon activity inhibiting properties and the IFNAR2 determinant binding properties described herein. Fv clones corresponding to these embodiments can be selected by using combinations of phage display library screening procedures for selection of clones with unique type I interferon activity inhibition profiles and phage display library screening procedures for selection of clones with unique IFNAR2 determinant binding properties.

For example, the invention provides anti-IFNAR2 antibodies and Fv clones which bind to one or more of amino acid positions 133, 134, 135, and 139 in situ in the sequence of IFNAR2, which bind to one or more of amino acids 153, 154 and 156 in situ in the sequence of IFNAR2, which block the binding of a first type I interferon to IFNAR2, and which do not block the binding of a second type I interferon to IFNAR2. Such Fv clones can be selected by (1) isolating anti-IFNAR2 clones from a phage library as described in Section B(I)(2) above, and optionally amplifying the isolated population of phage clones by growing up the population in a suitable bacterial host; (2) selecting a first type I interferon and a second type I interferon against which blocking and non-blocking activity, respectively, is desired; (3) adsorbing the anti-IFNAR2 phage clones to immobilized IFNAR2; (4) using an excess of the first type I interferon to elute the adsorbed clones that recognize IFNAR2-binding determinants which overlap or are shared with the IFNAR2-binding determinants of the first type I interferon; (5) readsorbing the clones isolated from step (4) to immobilized IFNAR2; (6) using an excess of the second type I interferon to elute any undesired clones that recognize IFNAR2-binding determinants which overlap or are shared with the IFNAR2-binding determinants of the second type I interferon; (7) eluting the clones which remain adsorbed following step (6); (8) adsorbing the eluted clones to an immobilized, mutant IFNAR2 containing an Ala substitution at amino acid positions 133, 134, 135, and 139 in the sequence of IFNAR2 in order to adsorb undesired clones which bind to determinants on IFNAR2 that do not overlap with the amino acid positions 133, 134, 135, or 139; (9) recovering the clones which fail to adsorb to immobilized, mutant IFNAR2 from the flow-through fractions in step (8); and (10) repeating steps (8) and (9) in order to screen the recovered clones for non-adsorption to the corresponding immobilized, Ala-substituted mutant IFNAR2 for amino acid positions 153, 154 and 156.

In a preferred embodiment, the anti-IFNAR2 Fv clone is selected by using a second type I interferon that is IFN-β in steps (1)-(10) of the above procedure.

In another preferred embodiment, the anti-IFNAR2 Fv clone is selected by using a first type I interferon that is selected from the group consisting of IFN-αB, IFN-αG, IFN-αA, and IFN-αD and using a second type I interferon that is IFN-β in steps (1)-(10) of the above. procedure.

In another example, the invention provides anti-IFNAR2 antibodies and Fv clones which bind to one or more of amino acid positions 133, 134, 135, and 139 in situ in the sequence of IFNAR2, which bind to one or more of amino acids 153, 154 and 156 in situ in the sequence of IFNAR2, which block the binding of more than one first type I interferon to IFNAR2, and which do not block the binding of a second type I interferon to IFNAR2. Such Fv clones can be selected by (1) isolating anti-IFNAR2 clones from a phage library as described in Section B(I)(2) above, and optionally amplifying the isolated population of phage clones by growing up the population in a suitable bacterial host; (2) selecting the first type I interferons and the second type I interferon against which blocking and non-blocking activity, respectively, is desired; (3) adsorbing the anti-IFNAR2 phage clones to immobilized IFNAR2; (4) using an excess of one of the first type I interferons to elute the adsorbed clones that recognize IFNAR2-binding determinants which overlap or are shared with the IFNAR2-binding determinants of the first type I interferon; (5) readsorbing the clones isolated from step (4) to immobilized IFNAR2; (6) repeating steps (4) and (5) for each of the remaining type I interferons against which blocking activity is desired; (7) using an excess of the second type I interferon to elute any undesired clones that recognize IFNAR2-binding determinants which overlap or are shared with the IFNAR2-binding determinants of the second type I interferon; (8) eluting the clones which remain adsorbed following step (7); (9) adsorbing the eluted clones to an immobilized, mutant IFNAR2 containing an Ala substitution at amino acid positions 133, 134, 135, and 139 in the sequence of IFNAR2 in order to adsorb undesired clones which bind to determinants on IFNAR2 that do not overlap with the amino acid positions 133, 134, 135, or 139; (10) recovering the clones which fail to adsorb to the immobilized, mutant IFNAR2 from the flow-through fractions in step (9); and (11) repeating steps (9) and (10) in order to screen the recovered clones for non-adsorption to the corresponding immobilized, Ala-substituted mutant IFNAR2 for amino acid positions 153, 154 and 156.

In a preferred embodiment, the anti-IFNAR2 Fv clone is selected by using a second type I interferon that is IFN-β in steps (1)-( 11) of the above procedure.

In another preferred embodiment, the anti-IFNAR2 Fv clone is selected by using all of IFN-αB, IFN-αG, IFN-αA, and IFN-αD as the first type I interferons and using IFN-β as the second type I interferon in steps (1)-(11) of the above procedure.

II. Methods of Making Anti-IFNAR2 Hybridomas

The anti-IFNAR2 antibodies of the invention are preferably monoclonal. Also encompassed within the scope of the invention are Fab, Fab′, Fab′-SH and F(ab′)₂ fragments of the anti-IFNAR2 antibodies provided herein. These antibody fragments can be created by traditional means, such as enzymatic digestion, or may be generated by recombinant techniques. Such antibody fragments may be chimeric or humanized. These fragments are useful for the diagnostic and therapeutic purposes set forth below.

Monoclonal antibodies are obtained from a population of substantially homogeneous antibodies, ie., the individual antibodies comprising the population are identical except for possible naturally occurring mutations that may be present in minor amounts. Thus, the modifier “monoclonal” indicates the character of the antibody as not being a mixture of discrete antibodies.

The anti-IFNAR2 monoclonal antibodies of the invention can be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized to elicit lymphocytes that produce or are capable of producing antibodies that will specifically bind to the protein used for immunization. Antibodies to IFNAR2 generally are raised in animals by multiple subcutaneous (sc) or intraperitoneal (ip) injections of IFNAR2 and an adjuvant. In one embodiment, animals are immunized with a derivative of IFNAR2 that contains the extracellular domain (ECD) of IFNAR2 fused to the Fc portion of an immunoglobulin heavy chain. In a preferred embodiment, animals are immunized with an IFNAR2-IgG1 fusion protein as described in the Example below. Animals ordinarily are immunized against immunogenic conjugates or derivatives of IFNAR2 with monophosphoryl lipid-A (MPL)/trehalose dicrynomycolate (TDM) (Ribi Immunochem. Research, Inc., Hamilton, Mont.) and the solution is injected intradermally at multiple sites. Two weeks later the animals are boosted. 7 to 14 days later animals are bled and the serum is assayed for anti-IFNAR2 titer. Animals are boosted until titer plateaus.

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against IFNAR2. Preferably, the binding specificity of monoclonal antibodies produced by hybridoma cells is determined by immunoprecipitation or by an in vitro binding assay, such as radioimmunoassay (RIA) or enzyme-linked immunoadsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies:Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Anti-IFNAR2 antibodies of the invention possessing the unique properties described in Section B(I) above can be obtained by screening anti-IFNAR2 hybridoma clones for the desired properties by any convenient method. For example, if an anti-IFNAR2 monoclonal antibody that blocks or does not block the binding of certain type I interferons to IFNAR2 is desired, the candidate antibody can be tested in a binding competition assay, such as a competitive binding ELISA, wherein plate wells are coated with IFNAR2, and a solution of antibody in an excess of the type I interferon of interest is layered onto the coated plates, and bound antibody is detected enzymatically, e.g. contacting the bound antibody with HRP-conjugated anti-Ig antibody or biotinylated anti-Ig antibody and developing the HRP color reaction., e.g. by developing plates with streptavidin-HRP and/or hydrogen peroxide and detecting the HRP color reaction by spectrophotometry at 490 nm with an ELISA plate reader.

In another embodiment, the invention provides anti-IFNAR2 monoclonal antibodies that inhibit the anti-viral activity of a first type I interferon and do not inhibit the anti-viral activity of a second type I interferon. Any convenient type I interferon viral infectivity inhibition assay is suitable for use herein. Such assays are well known in the art, and include, for example, type I interferon-induced inhibition of encephalomyocarditis virus (EMC) infectivity in A549 cells as described in Current Protocols in Immunology, Coligan, J. E., Kruisbeek, A. M., Margulies, D. H., Shevach, E. M., and Strober, W., eds, Greene Publishing Associates and Wiley-Interscience, (1992), vol. 2, unit 6.9.1. Generally, cells are seeded in attached cell culture plates, grown for 1 day, and then incubated for an additional day in the presence of a predetermined number of units of a selected type I interferon plus various concentrations of the candidate anti-IFNAR2 antibody. Culture supernatants are then removed and cells are challenged with virus and incubated for an additional day. The candidate anti-IFNAR2 antibody that inhibits the anti-viral activity of a selected type I interferon will inhibit more anti-viral activity than the baseline level of anti-viral activity inhibition measured in the presence of an equivalent concentration of control antibody. Optionally, the candidate anti-IFNAR2 antibody that inhibits the anti-viral activity of a selected type I interferon will inhibit at least at or about 30%, or at least at or about 50%, or at least at or about 70%, or at least at or about 80%, or at least at or about 90%, or at least at or about 95%, or at or about 100% of the activity of the type I interferon in the anti-viral activity assay as compared to baseline activity measured in the presence of an equivalent concentration of control antibody. The candidate anti-IFNAR2 antibody that does not inhibit the anti-viral activity of a selected type I interferon will exhibit similar or approximately the same level of anti-viral activity inhibition as a control antibody.

In a preferred embodiment, each type I interferon species used in the viral infectivity assay is titrated to a concentration that provides the same level of inhibition of viral activity as that induced by a preselected number of units of an IFN-a standard. This concentration serves to provide the normalized units of the subject type I interferon species. In order to assess the ability of an anti-IFNAR2 antibody to inhibit the anti-viral activity of various type I interferons, the effective concentration (EC50) of anti-IFNAR2 antibody for inhibiting 50% of a particular type I interferon's anti-viral activity (at the concentration titrated to provide the normalized units of activity) is determined for each type I interferon to be tested. In another preferred embodiment, each type I interferon to be tested is normalized to at least at or about 1 unit/ml, or at or about 1 unit/ml to at or about 1,000 units/ml, or at or about 1 unit/ml to at or about 100 units/ml, of human IFN-α2. In yet another preferred embodiment, each type I interferon to be tested is normalized to 100 units/ml of the NIH reference standard for recombinant human IFN-α2 (IFN-αA).

In still another preferred embodiment, the candidate anti-IFNAR2 antibody that does not inhibit the anti-viral activity of a selected type I interferon will exhibit no anti-viral effect at a concentration of at least at or about 1 μg/ml, or at least at or about 10 μg/ml, or at least at or about 20 μg/ml, or at least at or about 30 μg/ml, or at least at or about 50 μg/ml, or at least at or about 100 μg/ml, in the inhibition of EMC infectivity in A549 cells assay described in Current Protocols in Immunology, Coligan, J. E., Kruisbeek, A. M., Margulies, D. H., Shevach, E. M., and Strober, W., eds, Greene Publishing Associates and Wiley-Interscience, (1992), vol. 2, unit 6.9.1, wherein each type I interferon is normalized to 100 units/ml of NIH reference standard for recombinant human IFN-α2 (IFN-αA).

In one aspect, the invention provides anti-IFNAR2 monoclonal antibodies that inhibit the anti-viral activity of a first type I interferon and do not inhibit the anti-viral activity of IFN-αD.

In another aspect, the invention provides anti-IFNAR2 monoclonal antibodies that inhibit the anti-viral activity of a first type I interferon and do not inhibit the anti-viral activity of IFN-αA.

In yet another aspect, the invention provides anti-IFNAR2 monoclonal antibodies that inhibit the anti-viral activity of a first type I interferon and do not inhibit the anti-viral activity of IFN-αB.

In still another aspect, the invention provides anti-IFNAR2 monoclonal antibodies that inhibit the anti-viral activity of a first type I interferon and do not inhibit the anti-viral activity of IFN-α_{II}1.

In a further aspect, the invention provides anti-IFNAR2 monoclonal antibodies that inhibit the anti-viral activity of a first type I interferon and do not inhibit the anti-viral activity of IFN-β.

In an additional aspect, the invention provides anti-IFNAR2 monoclonal antibodies that inhibit the anti-viral activity of a first type I interferon selected from the group consisting of IFN-αD, IFN-αA, IFN-αG and IFN-αB and do not inhibit the anti-viral activity of IFN-β.

If an anti-IFNAR2 monoclonal antibody that binds to a particular IFNAR2 determinant(s) is desired, the candidate antibody can be screened for the presence or absence of differential affinity to wild type IFNAR2 and to mutant IFNAR2 that contains Ala substitution(s) at the determinant(s) of interest as described above. In one aspect, the candidate antibody can be tested for binding to wild type IFNAR2 and mutant IFNAR2 in an immunoprecipitation or immunoadsorption assay. For example, a capture ELISA can be used wherein plates are coated with a given concentration of wild type IFNAR2 or an equal concentration of mutant IFNAR2, the coated plates are contacted with equal concentrations of the candidate antibody, and the bound antibody is detected enzymatically, e.g. contacting the bound antibody with HRP-conjugated anti-Ig antibody and developing the HRP color reaction. The candidate antibody that binds to the particular IFNAR2 determinant(s) of interest will exhibit binding activity with wild type IFNAR2 that is greater than the candidate antibody's binding activity with the corresponding Ala-substituted IFNAR2 mutant (i.e. a binding level with wild type IFNAR2 that is above the background binding level with mutant IFNAR2). Optionally, the candidate antibody that binds to the particular IFNAR2 determinant(s) of interest will exhibit binding activity with the corresponding Ala-substituted IFNAR2 mutant that is less than about 50%, or less than about 30%, or less than about 20%, or less than about 10%, or less than about 7%, or less than about 6%, or less than about 5%, or less than about 4%, or less than about 3%, or less than about 2%, or less than about 1%, or about 0% of the antibody's binding activity with wild type IFNAR2, e.g. as determined by dividing the HRP color reaction optical density observed for capture ELISA with IFNAR2 mutant adsorbent by the HRP color reaction optical density observed for capture ELISA with wild type IFNAR2 adsorbent.

In other embodiments, the invention provides anti-IFNAR2 antibodies which possess combinations of the type I interferon activity inhibiting and the IFNAR2 determinant binding properties described herein. Anti-IFNAR2 antibodies corresponding to these embodiments can be obtained by using combinations of the type I interferon competitive binding and/or activity inhibition assays described above for selection of antibodies with unique type I interferon inhibiting properties and the immunoprecipitation or immunoadsorption screening procedures described above for selection of antibodies with unique IFNAR2 determinant binding properties.

In one example, the invention provides an anti-IFNAR2 antibody that binds to one or more of amino acid positions 133, 134, 135, and 139 in situ in the sequence of IFNAR2, binds to one or more of amino acids 153, 154 and 156 in situ in the sequence of IFNAR2, inhibits the anti-viral activity of a first type I interferon, and does not inhibit the anti-viral activity of a second type I interferon.

In another example, the invention provides an anti-IFNAR2 antibody that binds to one or more of amino acid positions 133, 134, 135, and 139 in situ in the sequence of IFNAR2, binds to one or more of amino acids 153, 154 and 156 in situ in the sequence of IFNAR2, inhibits the anti-viral activity of a first type I interferon, and does not inhibit the anti-viral activity of IFN-β.

In still another example, the invention provides an anti-IFNAR2 antibody that binds to one or more of amino acid positions 133, 134, 135, and 139 in situ in the sequence of IFNAR2, binds to one or more of amino acids 153, 154 and 156 in situ in the sequence of IFNAR2, inhibits the anti-viral activity of a first type I interferon selected from the group consisting of IFN-αD, IFN-αA, IFN-αG and IFN-αB, and does not inhibit the anti-viral activity of IFN-β.

In yet another example, the invention provides an anti-IFNAR2 antibody that binds to one or more of amino acid positions 133, 134, 135, and 139 in situ in the sequence of IFNAR2, binds to one or more of amino acids 153, 154 and 156 in situ in the sequence of IFNAR2, inhibits the anti-viral activity of more than one type I interferon, and does not inhibit the anti-viral activity of another type I interferon.

In a further example, the invention provides an anti-IFNAR2 antibody that binds to one or more of amino acid positions 133, 134, 135, and 139 in situ in the sequence of IFNAR2, binds to one or more of amino acids 153, 154 and 156 in situ in the sequence of IFNAR2, inhibits the anti-viral activity of more than one type I interferon, and does not inhibit the anti-viral activity of IFN-β.

In an additional example, the invention provides an anti-IFNAR2 antibody that binds to one or more of amino acid positions 133, 134, 135, and 139 in situ in the sequence of IFNAR2, binds to one or more of amino acids 153, 154 and 156 in situ in the sequence of IFNAR2, inhibits the anti-viral activity of IFN-αD, IFN-αA, IFN-αG and IFN-αB, and does not inhibit the anti-viral activity of IFN-β.

In another embodiment, the invention provides the anti-IFNAR2 monoclonal antibody produced by hybridoma cell line 1F3 (ATCC Deposit No.).

In yet another embodiment, the invention provides the anti-IFNAR2 monoclonal antibody produced by hybridoma cell line 3B7 (ATCC Deposit No. ).

In an additional embodiment, the invention provides the anti-IFNAR2 monoclonal antibody produced by hybridoma cell line 1D3 (ATCC Deposit No. ).

In still another embodiment, the invention provides anti-IFNAR2 monoclonal antibodies that compete with 1F3 antibody, 3B7 antibody or 1D3 antibody for binding to IFNAR2. Such competitor antibodies include antibodies that recognize an IFNAR2 epitope that is the same as or overlaps with the IFNAR2 epitope recognized by an antibody selected from the group consisting of the 1F3, 3B7 and 1D3 antibodies. Such competitor antibodies can be obtained by screening anti-IFNAR2 hybridoma supernatants for binding to immobilized IFNAR2 in competition with labeled 1F3 antibody, 3B7 antibody or 1D3 antibody. A hybridoma supernatant containing competitor antibody will reduce the amount of bound, labeled antibody detected in the subject competition binding mixture as compared to the amount of bound, labeled antibody detected in a control binding mixture containing irrelevant (or no) antibody. Any of the competition binding assays described in Section IV below are suitable for use in the foregoing procedure.

In another aspect, the invention provides an anti-IFNAR2 monoclonal antibody that comprises the complementarity determining regions (CDRS) of the 1F3 antibody. In yet another aspect, the invention provides an anti-IFNAR2 monoclonal antibody that comprises the complementarity determining regions (CDRS) of the 3B7 antibody. In still another aspect, the invention provides an anti-IFNAR2 monoclonal antibody that comprises the complementarity determining regions (CDRs) of the ID3 antibody. An anti-IFNAR2 monoclonal antibody that comprises the CDRs of 1F3, 3B7 or 1D3 can be constructed by isolating and cloning DNA encoding the variable regions of the 1F3, 3B7 or 1D3 antibody, identifying the CDRs of the 1F3, 3B7 or 1D3 parental antibody, grafting the CDRs onto a template antibody sequence, e.g. a human antibody sequence which is closest to the corresponding murine sequence of the parental antibody, or a consensus sequence of all human antibodies in the particular subgroup of the parental antibody light or heavy chain, and expressing the resulting chimeric light and/or heavy chain variable region sequence(s), with or without accompanying constant region sequence(s), in recombinant host cells as described in Section III below.

III. Methods of Constructing Recombinant Anti-IFNAR2 Antibodies

DNA encoding the hybridoma-derived monoclonal antibodies or phage display Fv clones of the invention is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immnunoglobulin protein, to obtain the synthesis of the desired monoclonal antibodies in the recombinant host cells. Review articles on recombinant expression in bacteria of antibody-encoding DNA include Skerra et al., Curr. Opinion in Immunol, 5: 256 (1993) and Pluckthun, Immunol. Revs, 130: 151(1992).

DNA encoding the Fv clones of the invention can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. A Fv clone derived from the variable domain DNA of one animal (such as human) species and then fused to constant region DNA of another animal species to form coding sequence(s) for “hybrid”, full length heavy chain and/or light chain is included in the definition of “chimeric” and “hybrid” antibody as used herein. In a preferred embodiment, a Fv clone derived from human variable DNA is fused to human constant region DNA to form coding sequence(s) for all human, full or partial length heavy and/or light chains.

DNA encoding anti-IFNAR2 antibody derived from a hybridoma of the invention can also be modified, for example, by substituting the coding sequence for human heavy- and light-chain constant domains in place of homologous murine sequences derived from the hybridoma clone (e.g. as in the method of Morrison et al., Proc. Natl. Acad. Sci. USA, 81: 6851-6855 (1984)). DNA encoding a hybridoma or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the invention.

Typically, such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for IFNAR2 and another antigen-combining site having specificity for a different antigen.

Chimeric or hybrid antibodies also can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate.

a. Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. It will be appreciated that variable domain sequences obtained from any non-human animal phage display library-derived Fv clone or from any non-human animal hybridoma-derived antibody clone provided as described herein can serve as the “import” variable domain used in the construction of the humanized antibodies of the invention. Humanization can be essentially performed following the method of Winter and co-workers (Jones et al., Nature, 321:,522 (1986); Riechmann et al., Nature, 332: 323 (1988); Verhoeyen et al., Science, 239: 1534 (1988)), by substituting non-human animal, e.g. rodent, CDRs or CDR sequences for the corresponding sequences of a human antibody. Accordingly, such “humanized” antibodies are chimeric antibodies (Cabilly et al., supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in non-human animal, e.g. rodent, antibodies.

The choice of human variable domains, both light and heavy., to be used in making the humanized antibodies is very important to reduce antigenicity. According to the so-called “best-fit” method, the sequence of the variable domain of a non-human animal, e.g. rodent, antibody is screened against the entire library of known human variable-domain sequences. The human sequence which is closest to that of the non-human animal is then accepted as the human framework (FR) for the humanized antibody (Sims et al, J. Immunol., 151: 2296 (1993); Chothia and Lesk, J. Mol. Biol., 196: 901 (1987)). Another method uses a particular framework derived from the consensus sequence of all human antibodies of a particular subgroup light or heavy chains. The same framework can be used for several different humanized antibodies (Carter et al., Proc. Natl. Acad. Sci USA, 89: 4285 (1992); Presta et al., J. Immunol., 151: 2623 (1993)).

It is also important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind to its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

b. Human Antibodies

Human anti-IFNAR2 antibodies of the invention can be constructed by combining Fv clone variable domain sequence(s) selected from human-derived phage display libraries with known human constant domain sequences(s) as described above. Alternatively, human monoclonal anti-IFNAR2 antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is now possible to produce transgenic animals (e.g. mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci USA, 90: 2551 (1993); Jakobovits et al., Nature, 362: 255 (1993); Bruggermann et al., Year in Immunol., 20 7: 33 (1993).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described above is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

c. Bispecific Antibodies

Bispecific antibodies are monoclonal, preferably human or humanized, antibodies that have binding specificities for at least two different antigens. In the present case, one of the binding specificities is for IFNAR2 and the other is for any other antigen. Exemplary bispecific antibodies may bind to two different epitopes of the IFNAR2 protein. Bispecific antibodies may also be used to localize cytotoxic agents to cells which express IFNAR2. These antibodies possess an IFNAR2-binding arm and an arm which binds the cytotoxic agent (e.g. saporin, anti-interferon-a, vinca alkaloid, ricin A chain, methotrexate or radioactive isotope hapten). Bispecific antibodies can be prepared as full length antibodies or antibody fragments (e.g. F(ab′)₂bispecific antibodies).

Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature, 305: 537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one armn, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach, the interface between a pair of antibody molecules can be engineered to maximize the percentage of heterodimers which are recovered from recombinant cell culture. The preferred interface comprises at least a part of the CH³ domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/00373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′-fragments generated are then converted to thionitrobenzoate (TNB) derivatives. One of the Fab′ TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol, 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol 147: 60 (1991).

IV. Diagnostic Uses of Anti-IFNAR2 Antibodies

The anti-IFNAR2 antibodies of the invention are unique research reagents which provide anti-type I interferon activity templates for use in chemical library screening, wherein the practitioner can use a signal transduction assay as an initial, high volume screen for agents that exhibit an anti-type I interferon activity pattern that is similar to the anti-type I interferon activity pattern of an anti-IFNAR2 antibody of the invention. In this way, candidate agents likely to exhibit a desired type I interferon activity inhibition profile can be obtained with ease, avoiding prohibitively expensive and logistically impossible numbers of type I interferon induced viral inhibition assays or cell proliferation inhibition assays on large chemical libraries.

In one embodiment, the anti-IFNAR2 antibodies of the invention are used to screen chemical libraries in a Kinase Receptor Activation (KIRA) Assay as described in WO 95/14930 (published 1 June 1995). The KIRA assay is suitable for use herein because ligand binding to the type I interferon receptor complex in situ in on the surface of host cells expressing the receptor induces a rapid increase in the phosphorylation of tyrosine residues in the intracellular domains of both IFNAR1 and IFNAR2 components of the receptor as taught in Platanias and Colamonici, J. Biol. Chem., 269: 17761-17764 (1994). The level of tyrosine phosphorylation can be used as a measure of signal transduction. The effect of an anti-IFNAR2 antibody of the invention on the levels of tyrosine phosphorylation induced by various type I interferons in the KIRA assay can be used as a bench mark activity pattern for comparison to the activity patterns generated by the library compounds in the assay.

The KIRA assay suitable for use herein employs a host cell that expresses the type I interferon receptor (both IFNARl and IFNAR2 components of the receptor) and the particular series of type I interferons which define the inhibitor profile of interest. Cells which naturally express the human type I interferon receptor, such as the human Daudi cells and U-266 human myeloma cells described in Colamonici and Domanski, J. Biol. Chem. 268: 10895-10899 (1993), can be used. In addition, cells which are transfected with the IFNAR1 and IFNAR2 components and contain intracellular signaling proteins necessary for type I interferon signal transduction, such as mouse L-929 cells as described in Domanski et al., J. Biol. Chem., 270: 21606-21611 (1995), can be used. In the KIRA assay, the candidate antagonist is incubated with each type I interferon ligand to be tested, and each incubation mixture is contacted with the type I interferon receptor-expressing host cells. The treated cells are lysed, and IFNAR2 protein in the cell lysate is immobilized by capture with solid phase anti-IFNAR2 antibody. Signal transduction is assayed by measuring the amount of tyrosine phosphorylation that exists in the intracellular domain (ICD) of captured IFNAR2 and the amount of tyrosine phosphorylation that exists in the intracellular domain of any co-captured IFNAR1. Alternatively, cell lysis and immunoprecipitation can be performed under denaturing conditions in order to avoid co-capture of IFNAR1 and permit measurement of IFNAR2 tyrosine phosphorylation alone, e.g. as described in Platanias et al., J. Biol. Chem., 271: 23630-23633 (1996). The level of tyrosine phosphorylation can be accurately measured with labeled anti-phosphotyrosine antibody which identifies phosphorylated tyrosine residues.

In another embodiment, a host cell coexpressing IFNAR1 and a chimeric construct containing IFNAR2 fused at its carboxy terminus to an affinity handle polypeptide is used in the KIRA assay. The chimeric IFNAR2 construct permits capture of the construct from cell lysate by use of a solid phase capture agent (in place of an anti-IFNAR2 antibody) specific for the affinity handle polypeptide. In a preferred embodiment, the affinity handle polypeptide is Herpes simplex virus glycoprotein D (gD) and the capture agent is an anti-gD monoclonal antibody as described in Examples 2 and 3 of WO 95/14930.

In this system, the anti-IFNAR2 antibody of the invention that possesses the type I interferon inhibition activity profile of interest is used as a standard for analysis of the tyrosine phosphorylation patterns generated by the members of the chemical library that is screened. The IFNAR2 ICD tyrosine phosphorylation pattern generated by the anti-IFNAR2 antibody standard is compared to the tyrosine phosphorylation patterns produced in the library screen, and patterns found to match that of the anti-IFNAR2 antibody standard identify candidate agents that are likely to have a type I interferon activity inhibition profile similar to that of the anti-IFNAR2 antibody standard. Accordingly, the anti-IFNAR2 antibody of the invention provides a useful means to quickly and efficiently screen large chemical libraries for compounds likely to exhibit the particular type I interferon activity inhibition profile of the antibody.

In addition, the anti-IFNAR2 antibodies of the invention are useful in diagnostic assays for IFNAR2 expression in specific cells or tissues wherein the antibodies are labeled as described below and/or are immobilized on an insoluble matrix. Anti-IFNAR2 antibodies also are useful for the affinity purification of IFNAR2 from recombinant cell culture or natural sources.

Anti-IFNAR2 antibodies can be used for the detection of IFNAR2 in any one of a number of well known diagnostic assay methods. For example, a biological sample may be assayed for IFNAR2 by obtaining the sample from a desired source, admixing the sample with anti-IFNAR2 antibody to allow the antibody to form antibody/IFNAR2 complex with any IFNAR2 present in the mixture, and detecting any antibody/IFNAR2 complex present in the mixture. The biological sample may be prepared for assay by methods known in the art which are suitable for the particular sample. The methods of admixing the sample with antibodies and the methods of detecting antibody/IFNAR2 complex are chosen according to the type of assay used. Such assays include competitive and sandwich assays, and steric inhibition assays. Competitive and sandwich methods employ a phase-separation step as an integral part of the method while steric inhibition assays are conducted in a single reaction mixture.

Analytical methods for IFNAR2 all use one or more of the following reagents: labeled IFNAR2 analogue, immobilized IFNAR2 analogue, labeled anti-IFNAR2 antibody, immobilized anti-IFNAR2 antibody and steric conjugates. The labeled reagents also are known as “tracers.”

The label used is any detectable functionality that does not interfere with the binding of IFNAR2 and anti-IFNAR2 antibody. Numerous labels are known for use in immunoassay, examples including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carboduimides, dimaleimides, bis-imidates, bis-diazotized benzidine, and the like may be used to tag the antibodies with the above-described fluorescent, chemiluminescent, and enzyme labels. See, for example, U.S. Pat. Nos. 3,940,475 (fluorimetry) and 3,645,090 (enzymes); Hunter et al., Nature, 144: 945 (1962); David eta al., Biochemistry, 13: 1014-1021 (1974); Pain et al., J. Immunol. Methods, 40: 219-230 (1981); and Nygren, J. Histochem. and Cytochem., 30: 407-412 (1982). Preferred labels herein are enzymes such as horseradish peroxidase and alkaline phosphatase.

The conjugation of such label, including the enzymes, to the antibody is a standard manipulative procedure for one of ordinary skill in immunoassay techniques. See, for example, O'Sullivan et al., “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Immobilization of reagents is required for certain assay methods. Immobilization entails separating the anti-IFNAR2 antibody from any IFNAR2 that remains free in solution. This conventionally is accomplished by either insolubilizing the anti-IFNAR2 antibody or IFNAR2 analogue before the assay procedure, as by adsorption to a water-insoluble matrix or surface (Bennich et al., U.S. Pat. No. 3,720,760), by covalent coupling (for example, using glutaraldehyde cross-linking), or by insolubilizing the anti-IFNAR2 antibody or IFNAR2 analogue afterward, e.g., by immunoprecipitation.

Other assay methods, known as competitive or sandwich assays, are well established and widely used in the commercial diagnostics industry.

Competitive assays rely on the ability of a tracer IFNAR2 analogue to compete with the test sample IFNAR2 for a limited number of anti-IFNAR2 antibody antigen-binding sites. The anti-IFNAR2 antibody generally is insolubilized before or after the competition and then the tracer and IFNAR2 bound to the anti-IFNAR2 antibody are separated from the unbound tracer and IFNAR2. This separation is accomplished by decanting (where the binding partner was preinsolubilized) or by centrifuging (where the binding partner was precipitated after the competitive reaction). The amount of test sample IFNAR2 is inversely proportional to the amount of bound tracer as measured by the amount of marker substance. Dose-response curves with known amounts of IFNAR2 are prepared and compared with the test results to quantitatively determine the amount of IFNAR2 present in the test sample. These assays are called ELISA systems when enzymes are used as the detectable markers.

Another species of competitive assay, called a “homogeneous” assay, does not require a phase separation. Here, a conjugate of an enzyme with the IFNAR2 is prepared and used such that when anti-IFNAR2 antibody binds to the IFNAR2 the presence of the anti-IFNAR2 antibody modifies the enzyme activity. In this case, the IFNAR2 or its immunologically active fragments are conjugated with a bifunctional organic bridge to an enzyme such as peroxidase. Conjugates are selected for use with anti-IFNAR2 antibody so that binding of the anti-IFNAR2 antibody inhibits or potentiates the enzyme activity of the label. This methodper se is widely practiced under the name of EMIT.

Steric conjugates are used in steric hindrance methods for homogeneous assay. These conjugates are synthesized by covalently linking a low-molecular-weight hapten to a small IFNAR2 fragment so that antibody to hapten is substantially unable to bind the conjugate at the same time as anti-IFNAR2 antibody. Under this assay procedure the IFNAR2 present in the test sample will bind anti-IFNAR2 antibody, thereby allowing anti-hapten to bind the conjugate, resulting in a change in the character of the conjugate hapten, e.g., a change in fluorescence when the hapten is a fluorophore.

Sandwich assays particularly are useful for the determination of IFNAR2 or anti-IFNAR2 antibodies. In sequential sandwich assays an immobilized anti-IFNAR2 antibody is used to adsorb test sample IFNAR2, the test sample is removed as by washing, the bound IFNAR2 is used to adsorb a second, labeled anti-IFNAR2 antibody and bound material is then separated from residual tracer. The amount of bound tracer is directly proportional to test sample IFNAR2. In “simultaneous” sandwich assays the test sample is not separated before adding the labeled anti-IFNAR2. A sequential sandwich assay using an anti-IFNAR2 monoclonal antibody as one antibody and a polyclonal anti-IFNAR2 antibody as the other is useful in testing samples for IFNAR2.

The foregoing are merely exemplary diagnostic assays for IFNAR2. Other methods now or hereafter developed that use anti-IFNAR2 antibody for the determination of IFNAR2 are included within the scope hereof, including the bioassays described above.

V. Therapeutic Compositions and Administration of Anti-IFNAR2 Antibodies

Therapeutic formulations of the anti-IFNAR2 antibodies of the invention are prepared for storage by mixing antibody having the desired degree of purity with optional physiologically acceptable carriers, excipients, or stabilizers (Remington: The Science and Practice of Pharmacy, 19th Edition, Alfonso, R., ed, Mack Publishing Co. (Easton, Pa.: 1995)), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The anti-IFNAR2 antibody to be used for in vivo administration must be sterile. This is readily accomplished by filtration through sterile filtration membranes, prior to or following lyophilization and reconstitution. The anti-IFNAR2 antibody ordinarily will be stored in lyophilized form or in solution.

Therapeutic anti-IFNAR2 antibody compositions generally are placed into a container having a sterile access port, for example, an intravenous solution bag or vial having a stopper pierceable by a hypodermic injection needle.

The route of anti-IFNAR2 antibody administration is in accord with known methods, e.g. injection or infusion by intravenous, intraperitoneal, intracerebral, subcutaneous, intramuscular, intraocular, intraarterial, intracerebrospinal, or intralesional routes, or by sustained release systems as noted below. Preferably the antibody is given systemically.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman et al., Biopolymers, 22: 547-556 (1983)), poly (2-hydroxyethyl-methacrylate) (Langer et al., J. Biomed. Mater. Res., 15: 167-277 (1981) and Langer, Chem. Tech., 12: 98-105 (1982)), ethylene vinyl acetate (Langer et al., supra) or poly-D-(−)-3-hydroxybutyric acid (EP 133,988). Sustained-release anti-IFNAR2 antibody compositions also include liposomally entrapped antibody. Liposomes containing antibody are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamelar type in which the lipid content is greater than about 30 mol. % cholesterol, the selected proportion being adjusted for the optimal antibody therapy.

Anti-IFNAR2 antibody can also be administered by inhalation. Commercially available nebulizers for liquid formulations, including jet nebulizers and ultrasonic nebulizers are useful for administration. Liquid formulations can be directly nebulized and lyophilized powder can be nebulized after reconstitution. Alternatively, anti-IFNAR2 antibody can be aerosolized using a fluorocarbon formulation and a metered dose inhaler, or inhaled as a lyophilized and milled powder.

An “effective amount” of anti-IFNAR2 antibody to be employed therapeutically will depend, for example, upon the therapeutic objectives, the route of administration, the type of anti-IFNAR2 antibody employed, and the condition of the patient. Accordingly, it will be necessary for the therapist to titer the dosage and modify the route of administration as required to obtain the optimal therapeutic effect. Typically, the clinician will administer the anti-IFNAR2 antibody until a dosage is reached that achieves the desired effect. The progress of this therapy is easily monitored by conventional assays.

The patients to be treated with the anti-IFNAR2 antibody of the invention include preclinical patients or those with recent onset of immune-mediated disorders, and particularly autoimmune disorders. Patients are candidates for therapy in accord with this invention until such point as no healthy tissue remains to be protected from immune-mediated destruction. For example, a patient suffering from insulin-dependent diabetes mellitis (IDDM) can benefit from therapy with an anti-IFNAR2 antibody of the invention until the patient's pancreatic islet cells are no longer viable. It is desirable to administer an anti-IFNAR2 antibody as early as possible in the development of the immune-mediated or autoimmune disorder, and to continue treatment for as long as is necessary for the protection of healthy tissue from destruction by the patient's immune system. For example, the IDDM patient is treated until insulin monitoring demonstrates adequate islet response and other indicia of islet necrosis diminish (e.g. reduction in anti-islet antibody titers), after which the patient can be withdrawn from anti-IFNAR2 antibody treatment for a trial period during which insulin response and the level of anti-islet antibodies are monitored for relapse.

In the treatment and prevention of an immune-mediated or autoimmune disorder by an anti-IFNAR2 antibody, the antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the antibody, the particular type of antibody, the method of administration, the scheduling of administration, and other factors known to medical practitioners. The “therapeutically effective amount” of antibody to be administered will be governed by such considerations, and is the minimum amount necessary to prevent, ameliorate, or treat the disorder, including treating chronic autoimmune conditions and immunosuppression maintenance in transplant recipients. Such amount is preferably below the amount that is toxic to the host or renders the host significantly more susceptible to infections.

As a general proposition, the initial pharmaceutically effective amount of the antibody administered parenterally will be in the range of about 0.1 to 50 mg/kg of patient body weight per day, with the typical initial range of antibody used being 0.3 to 20 mg/kg/day, more preferably 0.3 to 15 mg/kg/day. The desired dosage can be delivered by a single bolus administration, by multiple bolus administrations, or by continuous infusion administration of antibody, depending on the pattern of pharmacokinetic decay that the practitioner wishes to achieve.

As noted above, however, these suggested amounts of antibody are subject to a great deal of therapeutic discretion. The key factor in selecting an appropriate dose and scheduling is the result obtained, as indicated above.

The antibody need not be, but is optionally formulated with one or more agents currently used to prevent or treat the immune-mediated or autoimmune disorder in question. For example, in rheumatoid arthritis, the antibody may be given in conjunction with a glucocorticosteroid. The effective amount of such other agents depends on the amount of anti-IFNAR2 antibody present in the formulation, the type of disorder or treatment, and other factors discussed above. These are generally used in the same dosages and with administration routes as used hereinbefore or about from 1 to 99% of the heretofore employed dosages.

Further details of the invention can be found in the following example, which further defines the scope of the invention. All references cited throughout the specification, and the references cited therein, are hereby expressly incorporated by reference in their entirety.

### EXAMPLE

Materials and Methods

Preparation of Soluble IFNAR2-IgG

A cDNA encoding the human immunoglobulin fusion proteins (immunoadhesins) based on the ECD of the hIFNAR2 (pRK5 hIFNAR2-IgG clone) was generated using methods similar to those described by Haak-Frendscho et al., Immunology 79: 594-599 (1993) for the construction of a murine IFN-γ receptor immunoadhesin. Briefly, the plasmid pRKCD4₂Fc₁ was constructed as described in Example 4 of WO 89/02922 (PCT/US88/03414 published Apr. 6, 1989). The cDNA coding sequence for the first 216 residues of the mature hIFNAR2 ECD shown in FIG. 5 was obtained from the published sequence (Novick et al., Cell, 77: 391-400 (1994)). The CD4 coding sequence in the pRKCD4₂Fc₁ was replaced with the hIFNAR2 ECD encoding cDNA to form the pRK5hIFNAR2-IgG clone. The nucleic acid sequence (SEQ ID NO. 25) and amino acid sequence (SEQ ID NO. 26) for the hIFNAR2 ECD-IgG encoding insert of the clone are shown in FIG. 5. hIFNAR2-IgG was expressed in human embryonic kidney 293 cells by transient transfection using a calcium phosphate precipitation technique. The immunoadhesin was purified from serum-free cell culture supernatants in a single step by affinity chromatography on a protein A-sepharose column as described in Haak-Frendscho et al. (1993), supra. Bound hIFNAR2-IgG was eluted with 0.1 M citrate buffer, pH 3.0, containing 20% (w/v) glycerol. The hIFNAR2-IgG purified was over 95% pure, as judged by SDS-PAGE.

Production of hIFN-α Subtypes

Standard cloning procedures described in Maniatis et al., Molecular Cloning: A Laboratory Manual, Cold Spring Harbor Laboratory Press, Cold Spring Harbor, N.Y. (1989) were used to construct plasmids that direct the translocation of the various species of hIFN-α into the periplasmic space of E. coli PCR reactions were performed on cDNA clones of the various subspecies of hIFN-α disclosed in Goeddel et al., Nature 290: 20-26 (1981) with Nsil and StyI restriction sites added to the primers. These PCR products were then subdloned into the corresponding sites of the expression vector pB0720 described in Cunningham et al., Science 243:1330-1336 (1989). The resulting plasmids placed production of the hIFN-α subtypes under control of the E. coli phoA promoter and the heat-stable enterotoxin II signal peptide as described in Chang et al., Gene 55: 189-196 (1987). The correct DNA sequence of each gene was confirmed using the United States Biochemical Sequenase Kit version 2.0. Each plasmid was transformed into the E. coli strain 27C7 (ATCC # 55244) and grown in 10 liter fermentors as described in Carter et al., Bio/Technology 10: 163-167 (1992). Human hIFNs were purified from E. coli paste containing each IFN-α by affinity chromatography. Bacterial cells were lysed, and the lysate was centrifuged at 10,000× g to remove debris. The supernatant was applied to an immunoaffinity column containing a mouse anti-hIFN-αB antibody (LI-1) that was obtained as described in Staehelin et al., Proc. Natl. Acad. Sci. 78:1848-1852 (1981). LI-1 was immobilized on controlled pore glass by a modification of the method of Roy et al., Journal of Chromatography, 303: 225-228 (1984). The bound interferon was eluted from the column with 0.1 M citrate, pH 3.0, containing 20% (w/v) glycerol. The purified IFN was analyzed by SDS-PAGE and immunoblotting, and was assayed for bioactivity by the hIFN-induced anti-viral assay as described herein. HIFNβ was obtained from Sigma (St. Louis, Mo.).

Human IFN-α2/1 hybrid molecule (IFN-α2₁₋₆₂/α₁₆₄₋₁₆₆) was obtained as described in Rehberg et al., J. Biol. Chem. 257: 11497-11502 (1992) or Horisberger and Marco, Pharmac. Ther., 66: 507-534 (1995).

Generation of mAbs to hIFNAR2.

Balb/c mice were immunized into each hind foot pad 11 times at two week intervals, with 2.5 μg of hIFNAR2-IgG fusion protein resuspended in MPL-TDM (Ribi Immunochem. Research Inc., Hamilton, Mont.). Three days after the final boost, popliteal lymph node cells were fused with murine myeloma cells, P3X63AgU.1 (ATCC CRL1597), using 35% polyethylene glycol. Hybridomas were selected in HAT medium. Ten days after the fusion, hybridoma culture supernatants were first screened for mAbs binding to the hIFNAR2-IgG fusion protein but not to CD4-IgG in a capture ELISA. The selected culture supernatants further tested for their ability to block the ligand-receptor binding in a capture ELISA as described below and for their ability to recognize cell membrane receptors on U266 cells by flow cytometric analysis as described in Chuntharapai et al., J. Immunol., 152:1783-1789 (1994). After cloning the selected final hybridomas twice, their antigen specificity as well as blocking activities were confirmed in the ligand-receptor binding assay, IGSF-3 complex assay and anti-viral assay as described below.

Epitope Mapping Using a Competitive Binding ELISA

To determine whether the mAbs recognized the same or different epitopes, a competitive binding ELISA was performed as described in Kim et al., J. Immunol Method 156: 9-17 (1992), using biotinylated mAbs (Bio-mAb). mAbs were biotinylated using N-hydroxyl succinimide as described in Antibodies (A Laboratory Manual), Harlow, E. and Lane, D., eds, Cold Spring Harbor (1988), p. 341. Microtiter wells were coated with 50 μl of Goat anti-hIgG-Fc and kept overnight at 4° C., blocked with assay buffer for 1 hour, and incubated with 25 μl/well of IFNAR2-IgG (1 μg/ml) for 1 hour at room temperature. After washing microtiter wells, a mixture of a predetermined optimal concentration of Bio-mAb and a thousand-fold excess of unlabeled mAb was added into each well. Following 1 hour incubation at room temperature, plates were washed and the amount of Bio-mAb was detected by the addition of HRP-streptavidin. After washing the microtiter wells, the bound enzyme was detected by the addition of the substrate, and the plates were read at 490 nm with an ELISA plate reader.

Determination of the Affinities of mAbs

The equilibrium dissociation and association constant rates of anti-IFNAR2 mAbs were determined using KinExA™ automated immunoassay system (Sapidyne Instruments, Inc. Boise, Id.) modified as described in Blake et al., J. Biol. Chem., 271: 27677 (1996). Briefly, 1.0 ml of Anti-Human IgG Agarose beads (56 μm, Sigma. St. Louis, Mo.) were coated with 20 μg of IFNAR2-IgG in PBS by gentle mixing at room temperature (RT) for 1 hour. After washing with PBS, non specific binding sites were blocked by incubating with 10% human serum in PBS for 1 hour at RT. A bead pack (approximately 4 mm high) was created in the observation flow cell of the KinExA™ instrument. Briefly, the blocked beads were diluted into 30 ml of assay buffer (0.01% BSA/PBS). The diluted beads (550 μl) were drawn through the flow cell with a 20 micron screen and then washed with 1 ml of running buffer (0.01% BSA +0.05% Tween 20 in PBS). The beads were then disrupted gently with a brief backflush of running buffer, and allowed to set for 20 seconds in order to create a uniform and reproducible bead pack.

For equilibrium measurements, mAbs (5 ng/ml-31 pM-in 0.01% BSA/PBS)) were mixed with a serial dilutions of IFNAR2-IgG (concentrations from 2.5 nM to 5.0 pM) and incubated at RT for 2 minutes. Once equilibrium was reached, 4.5 ml of this mixture was drawn through the beads, followed by 250 μl of running buffer to wash out the unbound mAb. The primary mAbs bound to beads were detected by 1.5 ml of phycoerythrin (PE) labeled goat anti-mouse IgG drawn through the bead pack. Unbound labeled material was removed by drawing 4.5 ml of 0.5 M NaCl through the bead pack over a 3 minute period. The equilibrium constant was calculated by Scatchard analysis as described in Munson et al., Anal. Biochem., 160: 1085 (1980).

Electrophoretic Mobility Shift Assay (EMSA)

Briefly, 5 ng of α-IFNs plus various concentrations of anti-hIFNAR2 mAbs were incubated with 5×10⁵ Hela cells in 200 μl of DMEM for 30 minutes at 37° C. Cells were washed in PBS and resuspended in 125 μl buffer A (10 mM HEPES, pH 7.9, 10 mM KCL, 0.1 mM ETDA, 1 mM DTT, 1 mM phenylmethylsulfonyl floride, 10 μg/ml leupeptin, 10 μg/ml aprotinin) as described in Kurabayashi et al., Mol. Cell Biol., 15: 6386 (1995). After a 15 minute incubation on ice, cells were lysed by the addition of 0.025% NP40. The nuclear pellet was obtained by centrifugation and was resuspended in 50 μl buffer B (20 mM HEPES, pH 7.9, 400 mM NaCL, 0.1 mM EDTA, I mM DTT, 1 mM phenylmethylsulfonyl floride, 10 μg/ml leupeptin, 10 μg/ml aprotinin) and kept on ice for 30 min. The nuclear fraction was cleared by centrifugation and the supernatant stored at -70° C. until use. Double-stranded probes were prepared from single-stranded oligonucleotides (ISG15 top: 5′-GATCGGGAAAGGGAAACCGAAACTGAAGCC-3′ (SEQ ID NO. 1)), ISG15 bottom: 5′-GATCGGCTTCAGTTTCGGTTTCCCTTTCCC-3′ (SEQ ID NO. 2 )) utilizing a DNA polymerase I Klenow filling reaction with ³²P-dATP (3,000 Ci/mM, Amersham). Labeled oligonucleotides were purified from unincorporated radioactive nucleotides using BIO-Spin 30 columns (Bio-Rad). Binding reactions containing 5 μl nuclear extract, 25,000 cpm of labeled probe and 2 μg of non-specific competitor poly (dI-dC)-poly (dI-dC) in 15 μl binding buffer (10 mM Tris-HCL, pH 7.5, 50 mM NaCl, 1 mM EDTA, 1 mM DTT, 1 mM phenylmethylsulfonyl floride and 15% glycerol) were incubated at RT for 30 minutes. DNA-protein complexes were resolved in 6% non-denaturing polyacrylamide gels and analyzed by autoradiograph. The specificity of the assay was determined by the addition of 350 ng of unlabeled ISG15 probe in separate reaction mixtures. Formation of an ISGF3 specific complex was confirmed by a super shift assay with anti-STAT1 antibody.

Anti-viral Assay

The assay was done as described in Current Protocols in Immunol., Coligan, J. E., Kruisbeek, A. M., Margulies, D. H., Shevach, E. M., and Strober, W., eds, Greene Publishing Associates and Wiley-Interscience (1992), Vol. 2, Unit 6.9.1, using the human lung carcinoma cell line A549 challenged with encephalomyocarditis virus (EMC). Serial dilutions of mAbs were incubated with various units of type I IFNs in 50 μl DMEM at 37° C. These mixtures were then incubated with A549 cells (5×10⁵ cells/100 μl DMEM 4% FCS) for another 24 hours. Culture supernatants were then removed and challenged with 2×10⁵ pfu of encephalomyocarditis (EMC) virus in 100 μl DMEM with 2% FCS for an additional 24 hours. At the end of the incubation, cell viability was determined by visual microscopic examination. Cells were then incubated with seven 1:2 dilutions of mixtures containing anti-IFNAR2 mAbs plus type 1 hIFN for 24 hours. Each dilution was tested in duplicate. Cells were then washed and challenged with encephalomyocarditis (EMC) virus for another 24 hours. At the end of the experiment, the remaining viable cells were detected by crystal violet staining. The neutralizing antibody titer (EC50) was defined as the concentration of antibody which neutralizes 50% of the anti-viral cytophathic effect induced by 100 units/ml of type I IFNs. The units of type I IFNs used in this study were determined using NIH reference human IFN-α2 as a standard.

Generation of Various hIFNAR2-IgG Mutants

A cDNAs encoding residues 1-216 of the extracellular domain of IFNAR2 was constructed and expressed as an immunoadhesin. Single alanine substitution mutants were generated according to the method of Kunkel et al., Methods Enzymol. 154: 367-414 (1987), and Hebert et al., J. Biol. Chem., 268: 18549-18553 (1993). The plasmid DNA was isolated using an RPM Kit (BIO 101 Inc., La Jolla, Calif.) and was sequenced by the Sanger method using an ABI 373A DNA sequencer to verify the mutation. Mutant receptor-IgGs were expressed transiently in human 293 cells as described above. Transfected 293 cells were grown overnight in F-12:DMEM (50:50) containing 10% FCS, 2 mM glutamine, 100 μg/ml of penicillin, 100 μg/ml of streptomycin, 10 μg/ml of glycine, 15 μg/ml of hypoxanthine, and 5 μg/ml of thymidine, and then were placed in serum-free media. Three days later, culture supernatants were collected and used in a capture ELISA to determine the mAb binding sites.

Capture ELISA

Microtiter plates (Maxisorb; Nunc, Kamstrup, Denmark) were coated with 50 μl/well of 2 μg/ml of goat antibodies specific to the Fc portion of human IgG (Goat anti-hlgG-Fc, Cappel), in PBS, overnight at 4° C. and blocked with 2% BSA for 1 hour at room temperature. After washing the plate, 50 μl/well of 2 μg/ml of IFNAR2-IgG (or IFNAR2-IgG mutant) were added to each well for 1 hour. The remaining anti-Fc binding sites were blocked with PBS containing 3% human serum and 10 μg/ml of CD4-IgG for 1 hour. Plates were then incubated with 50 μl/well of 2 μg/ml of anti-IFNAR2 mAbs (or hybridoma culture supernatants) for 1 hour. Plates were then incubated with 50 μl/well of HRP-Goat anti-mouse IgG. The bound enzyme was detected by the addition of the TMB (3,3′,5,5′-tetramethylbenzidin) substrate, the reaction was stopped by the addition of the stop solution (Kirkegaard & Perry Lab, Gathersburg, Md.) and the plates were read at 450 nM with an ELISA plate reader. Between each step, plates were washed three times in wash buffer (PBS containing 0.05% Tween 20).

During the IFNAR2-IgG mutant analysis, the concentrations of immunoadhesin molecules in 293 transfected culture supernatants were determined using CD4-IgG as a standard and were adjusted to be equal to the lowest concentration (approximately 0.5 nm) of immunoadhesin molecules. The degree of mAb binding to these mutants was then compared to the wild type of the same concentration.

Results

mAbs to IFNAR2 Binding Different Epitopes Show Differential Blocking Activities

mAbs 1D3, 1F3 and 3B7, which recognize different epitopes on IFNAR2 by competitive binding ELISA using the molar ratio of biotinylated mAb to unlabeled mAb of 1:100 (Table 1 below), were developed to characterize the structure-function of IFNAR2. All of these mAbs belong to the IgG2a isotype and recognize membrane IFNAR2 on human myeloma U266 cells by FACS.

TABLE 1
Summary of the general characteristics of mAbs to IFNAR2

| | | | | Western^{e} | | Affinity^{e} |
|---|---|---|---|---|---|---|
| mAb | Isotype^{a} | FACS^{b} | Epitope^{c} | blot | Imm. ppt^{d} | Kd⁻¹ (pM) |
| 1D3 | IgG2a | +++ | A | + | ND | 242 |
| 1F3 | IgG2a | +++ | B | − | + | 5 |
| 3B7 | IgG2a | +++ | C | − | + | <1 |

^{a}The isotype of mAbs was determined using isotype specific goat anti-mouse Ig.
^{b}All these mAbs stained U266 human myeloma cells expressing IFNAR by FACS.
^{c}MAbs were shown to recognize different epitopes by competitive binding ELISA.
^{d}The immunoblotting was performed using IFNAR2 reduced with dithiothreitol (DTT).
^{e}U266 cells were biotinylated using NHS-LC-biotin and lysed with 1% NP-40. Biotinylated IFNAR2 were then precipitated by mAbs bound to protein-G-4B sepharose and separated on a 7.5% SDS-PAGE gel. Biotinylated IFNAR2 transferred onto nitrocellulose paper was then detected by HRP-streptavidin as described.
^{f}The affinity of mAbs were determined by Scatchard analysis using KINEX system.

Only mAb 1D3 recognizes the reduced IFNAR2 in the immunoblot, indicating that mAb 1D3 recognizes a linear epitope while others recognize conformational epitopes. The affinities (Kd⁻¹) of mAb 3B7, 1F3 and 1D3 were less than 1 pM, 5 pM and 242 pM, respectively, demonstrating that these are relatively high affinity mAbs

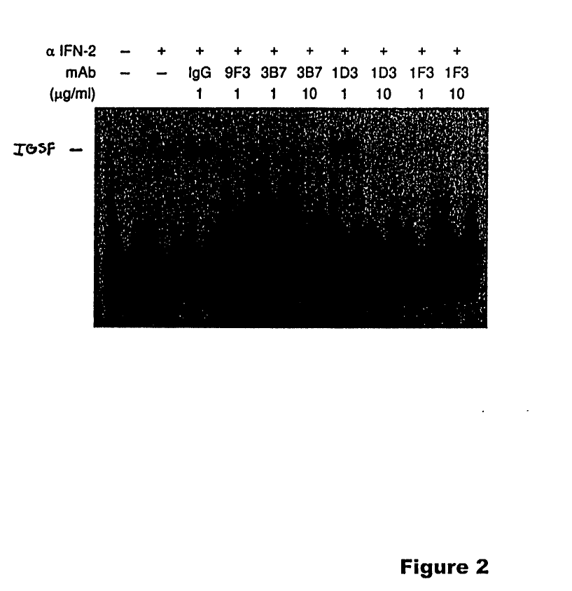

The neutralizing abilities of these mAbs were determined in the receptor-ligand binding ELISA (FIG. 1). At a concentration of 0.6 nM (0.1 μg/ml), mAbs 1F3 and 3B7 were able to block greater than 90% of the biotinylated IFNα-2/1 binding to IFNAR2-IgG while even at a concentration of 6 nM (1 μg/ml), mAb 1D3 showed no significant blocking activity. From these ELISA results, it was determined that mAbs 1F3 and 3B7 are blocking mAbs and that mAb 1D3 may not be a blocking mAb. To further determine blocking activities of these mAbs, mAbs were tested in the IGSF complex formation EMSA and in the anti-viral assay. FIG. 2 depicts the results obtained in IGSF complex formation induced by IFN-α2 (IFN-αA) using L929 cells expressing hIFNAR2. At a concentration of 1 μg/ml, mAbs 3B7 and 1F3, but not mAb 1D3, completely blocked the IGSF complex formation induced by human type I IFN-α2. At a concentration of 10 μg/ml, all three mAbs blocked the IGSF complex formation. These results demonstrated that all three mAbs are blocking mAbs but mAb 1D3 is a weak blocking mAb. Blocking activities of these mabs on the anti-viral activities of type 1 IFNs (IFNα-1(D), -2(A), -5(G), -8(B), -2/1 and IFN-β) are summarized in Table 2 below.

TABLE 2
Effects of anti-hIFNAR2 mAbs on the anti-viral effects of type 1 IFNs

| | EC50 of mAb (μg/ml) | | | | | |
|---|---|---|---|---|---|---|
| mAb | IFNα2/1 | IFNα1 | IFNα2 | IFNα5 | IFNα8 | IFNβ |
| 1D3 | 20 | 10 | 20 | 10 | 20 | NB |
| 1F3 | 3 | 2 | 3 | 1 | 3 | 2 |
| 3B7 | 0.6 | 0.1 | 1 | 0.1 | 0.3 | 0.3 |

The neutralizing antibody titer (EC50) was defined as the concentration of antibody which neutralizes 50% of the anti-viral cytopathic effects induced by 100 units/ml of type 1 IFNs on A549 cells. Experiment were done using serial dilutions of mAbs in the range of 0.1-30 μg/ml in duplicate. mAbs exhibiting no blocking effect at a concentration of 30 μg/ml in the assay were designated as nonblocking mAbs (NB).

mAbs 3B7 and 1F3 blocked the activities of all type 1 IFNs tested. The mAb 3B7 exhibited a blocking activity of 50% reduction in interferon-induced anti-viral cytophathic effects (EC50) at a concentration of less than 1 μg/ml on all type IFNs, and the EC50 concentration of mAb 1F3 was 5-20 fold higher than the EC50 concentration of mAb 3B7. Although mAb 1D3 was able to block the activity of all IFNa species except IFNβ, a much higher concentration of 1D3 antibody was required for inhibition of the IFN-α activities compared to mAbs 3B7 and 1F3.

Determination ofresidues on IFNAR2 which are important for the binding of IFNα-2/1 and neutralizing mAbs. In this study, hIFN-α2/1 hybrid molecule (IFN-α2₁₋₆₂/α1₆₄₋₁₆₆) was selected for testing due to its availability in a large quantity, its potent anti-viral activities in various animal species and its wide clinical use. The IFN-α2/1 hybrid molecule was generated by recombining IFN-α2(IFNαA) and IFN-α1 (IFN-αD), which are the most common IFN-α species produced in virus-infected leukocytes and which share a high degree of structural homology. The recombinant IFN-2/1 hybrid retains potent anti-viral activities in human as well as in mouse, confirming the presence of functional IFN receptor binding sites.

To determine areas of IFNAR2 which are important for the ligand binding, multiple and single alanine substitution mutants were generated as shown in Tables 3 and 4 below.

TABLE 3
Binding of IFN-α 2/1 and anti-IFNAR2 mAbs
to multiple alanine IFNAR2 mutants

| | | % Wild IFNAR2 binding | | | |
|---|---|---|---|---|---|
| residues | Alanine substitution | IFNα-2/1 | 1D3 | 1F3 | 3B7 |
| 7-11 | DYTDE (SEQ ID NO. 3) | 73 ± 10 | 105 ± 13 | 96 ± 1 | 101 ± 13 |
| | / AYTAA (SEQ ID NO. 4) | | | | |
| 29-33 | ELKNH (SEQ ID NO. 5) | 87 ± 22 | 80 ± 1 | 87 ± 6 | 82 ± 7 |
| | / ALANA (SEQ ID NO. 6) | | | | |
| 49-55 | KPEDLK (SEQ ID NO. 7) | 18 ± 2 | 39 ± 1 | 6 ± 0 | 4 ± 0 |
| | / APAALA (SEQ ID NO. 8) | | | | |
| 68-72 | DLTDE (SEQ ID NO. 9) | 16 ± 1 | 38 ± 2 | 5 ± 0 | 3 ± 0 |
| | / ALTAA (SEQ ID NO. 10) | | | | |
| 74-78 | RSTHE (SEQ ID NO. 11) | 16 ± 1 | 95 ± 1 | 16 ± 1 | 89 ± 2 |
| | / ASTAA (SEQ ID NO. 12) | | | | |
| 105-109 | DMSFE (SEQ ID NO. 13) | 19 ± 2 | 65 ± 1 | 8 ± 1 | 40 ± 1 |
| | / AMSFA (SEQ ID NO. 14) | | | | |
| 133-139 | EEELQFD (SEQ ID NO. 15) | 16 ± 1 | 5 ± 1 | 9 ± 0 | 35 ± 1 |
| | / AAALQFA (SEQ ID NO. 16) | | | | |
| 145-149 | EEQSE (SEQ ID NO. 17) | No expression | | | |
| | / AAQSA (SEQ ID NO. 18) | | | | |
| 153-157 | KKHKP (SEQ ID NO. 19) | 19 ± 1 | 14 ± 1 | 14 ± 0 | 35 ± 1 |
| | / AAHAP (SEQ ID NO. 20) | | | | |
| 159-163 | EIKGN (SEQ ID NO. 21) | 92 ± 5 | 89 ± 7 | 96 ± 5 | 93 ± 3 |
| | / AIAGN (SEQ ID NO. 22) | | | | |
| 172-173 | DK/AA | 73 ± 7 | 73 ± 5 | 91 ± 7 | 80 ± 3 |
| 187-192 | EHSDEA (SEQ ID NO. 23) | 82 ± 3 | 82 ± 10 | 83 ± 9 | 66 ± 10 |
| | / AASAAQ (SEQ ID NO. 24) | | | | |

Wild and mutant IFNAR2 adhesin molecules (0.5 nM) were captured with the goat anti-human IgG Fc reagent precoated onto ELISA plate wells. Biotinylated IFNα-A (50 nM) or mAbs (5 nM) were allowed to interact for 1 hour. After washing, the amounts of the ligand or mAbs bound were determined by the addition of HRP-streptavidin and HRP-sheep anti-mouse IgG, respectively.

TABLE 4
Binding of IFN-α 2/1 and anti-IFNAR2 mAbs to single alanine
IFNAR2 mutants

| | % Wild IFNAR2 binding | | | | |
|---|---|---|---|---|---|
| mutants | IFN-α_2/1 | 1D3 | 1F3 | 3B7 | Polyclonal |
| K49A | 68 ± 10 | 101 ± 1 | 104 ± 6 | 99 ± 1 | 98 ± 13 |
| E51A | 95 ± 9 | 103 ± 4 | 101 ± 4 | 67 ± 0 | 92 ± 3 |
| D52A | 97 ± 11 | 101 ± 1 | 107 ± 6 | 83 ± 2 | 94 ± 15 |
| K54A | 53 ± 1 | 87 ± 1 | 83 ± 2 | 87 ± 12 | 91 ± 13 |
| K57A | 113 | 110 | 120 | 91 | 120 |
| D68A | 15 ± 2 | 41 ± 1 | 8 ± 1 | 8 ± 0 | 38 ± 1 |
| D71A | 113 ± 26 | 100 ± 2 | 91 ± 3 | 110 ± 13 | 103 ± 12 |
| E72A | 129 ± 29 | 91 ± 7 | 106 ± 19 | 90 ± 13 | 94 ± 2 |
| R74A | 122 ± 28 | 92 ± 2 | 103 ± 12 | 93 ± 16 | 93 ± 13 |
| H77A | 83 ± 19 | 150 ± 4 | 12 ± 137 | 132 ± 16 | 150 ± 12 |
| E78A | 36 ± 3 | 92 ± 3 | 54 ± 4 | 94 ± 2 | 89 ± 14 |
| W101A | 22 ± 3 | 99 ± 4 | 90 ± 2 | 98 ± 2 | 100 ± 13 |
| I104A | 51 ± 5 | 104 ± 1 | 94 ± 6 | 104 ± 1 | 99 ± 13 |
| D105A | 53 ± 11 | 89 ± 3 | 62 ± 8 | 87 ± 2 | 97 ± 14 |
| E109A | 70 ± 9 | 106 | 121 | 111 | 100 ± 17 |
| E133A | 78 | 92 ± 3 | 96 ± 12 | 82 | 80 ± 12 |
| E134A | 86 ± 3 | 87 ± 1 | 82 ± 3 | 88 ± 12 | 92 ± 3 |
| E135A | 72 ± 2 | 88 ± 1 | 74 ± 1 | 80 ± 12 | 90 ± 1 |
| Q137A | 43 ± 4 | 63 ± 1 | 55 ± 4 | 61 ± 14 | 82 ± 1 |
| D139A | 62 | 87± | 100 ± 4 | 100 | 89 ± 14 |
| E145A | 78 ± 9 | 84 ± 2 | 82 ± 12 | 93 ± 12 | 91 ± 11 |
| E146A | 87 ± 4 | 93±± | 92 ± 3 | 98 ± 12 | 97 ± 5 |
| K153A | 71 ± 1 | 91 ± 1 | 86 ± 6 | 96 ± 11 | 93 ± 13 |
| K154A | 82 ± 18 | 90 ± 1 | 78 ± 6 | 89 ± 19 | 91 ± 4 |
| K156A | 62 | 83 ± 111 | 97 ± 110 | 87 ± 7 | 96 ± 17 |

Experiments were carried out as described in Table 3.

Upon analysis of the hydropathy profile of the IFNAR2, 12 charged areas in clusters of 2-7 residues were selected for substitution with alanines as shown in Table 3 above. Eleven out of 12 multiple mutants expressed as immunoadhesins as detected by anti-human IgG-Fc ELISA. IFNα-2/1 exhibited no binding to IFNAR2 multiple mutants in the residues of 49-55, 68-72, 74-78, 105-109, 133-139, 153-156 while multiple alanine substitutions in the area 7-11, 29-33, 159-163, 172-173 and 187-192 exhibited no effect on the binding of IFNα-2/1. No expression was obtained from the IFNAR2 multiple mutant containing alanine substitutions in residues 145-149 of IFNAR2, indicating that residues 145-149 are important for the integrity of IFNAR2-IgG. A computer model of IFNAR2 was constructed by displaying its sequence on the backbone of tissue factor (described in Muller et al., J. Mol. Biol., 256: 144-159 (1996)). FIG. 4 shows a three dimensional rendering of this model. According to the model, residues 49-55, 68-72, 74-78, 105-109, 133-139 and 153-156, are clustered into a major area. Thus, it appears that these areas of IFNAR2 contribute to the binding of IFN-α2/1. To confirm the integrity of these multiple substitution IFNAR2 mutants, the binding of anti-IFNAR2 mAbs to the mutants was also determined. All the mutants expressed exhibited 35-100% binding with at least one of these mAbs, compared to the wild type IFNAR2-IgG, indicating that these multiple mutants retain the general integrity of IFNAR2 structure. The data also demonstrated that the binding of the most potent blocking mAb 3B7 was dependent upon the residues 49-55 and 68-72 of IFNAR2, and was influenced by the residues 133-139 and 153-157. The epitopes recognized by the moderate blocking mAb 1F3 include residues 49-55, 68-72 and 74-78 in domain 1, and residues 105-109, 133-139 and 153-156 in domain 2. The critical areas recognized by mAb 1D3 are in residues 133-139 and 153-157 in domain 2 of IFNAR2 while residues 49-55 and 68-72 have some influence on mAb 1D3 binding.

To better define residues on IFNAR2 which play important roles in the binding of IFNα-2/1 and blocking mAbs, single alanine mutants were generated in the hydrophilic amino acids in residues of 49-156 (see Table 4 above). IFNAR2 single alanine mutants, D68A, E78A and W101A, exhibited no significant binding to IFN-α2/1 while IFNAR2 single alanine mutants I104A and D105A exhibited some binding to IFN-α2/1. Although no single residue which has crucial effects on the binding of the anti-IFNAR2 mAbs was detected, some of the multiple alanine substitution mutants shown in Table 3 exhibited a significant reduction in the binding of these mAbs. The experiments depicted in Tables 3 and 4 were done by capturing only 20-50 ng (0.2-0.5 nM) of mutant IFNAR2-IgG in goat anti-human IgG coated wells. After washing, the IFNAR2-IgG bound was detected by the addition of mAbs at a concentration of 1 μg/ml (6 nM) or biotinylated IFN-α2/1 at a concentration of 1 μg/ml (50 nM). Thus, the molar ratio between IFNAR2-IgG mutants captured and the mAb or type 1 IFN added is more than 1: 1000 fold. Under such experimental conditions, it was possible to detect almost all IFNAR2-IgG mutant bound. Similar data were obtained from five independent experiments. In order to confirm the findings shown in Table 3, the binding capacities of some of important IFNAR2-IgG mutants were determined using various concentration of IFN-α2/1 and mAb 1F3 (FIGS. 3A-3B). The binding of IFN-α2/1 to IFNAR2-IgG mutants D68, E78 and W101 at IFN-α2/1 concentrations of 5-50 nM was found to be less than 10% of binding to wild type IFNAR2-IgG while the EC 50 of mAb 1F3 for the binding to IFNAR2-IgG mutants I104A and D105A was determined to be approximately 10 fold higher than the EC50 of mAb 1 F3 for wild type IFNAR2-IgG. These results confirmed that residues D68, E78 and W101 play a crucial role in the binding of IFN-α2/1 and residues I104 and D105 influence the binding of IFN-α2/1. Among all of the single mutants tested, mutant E78A exhibited the biggest reduction (54%) in binding to mAb 1F3 as compared to wild type. The EC50 concentrations exhibited by mAb 1 F3 with respect to wild type IFNAR2-IgG, multiple 74-78 residue mutant (RSTHE(SEQ ID NO. 11 )/ASTAA(SEQ ID NO. 12)) and E78A mutant were determined to be 0.09 nM, 90 nM and 1.1 nM, respectively. These results indicate that the residue E78 plays some role on the binding of mAb 1F3 but the cluster of R74, H77 and E78 together has a drastic effect (1000 fold reduction) on the binding of mAb 1 F3 to IFNAR2-IgG.

Discussion

On the basis of structural homology, hematopoetic superfamily receptors can be divided into two classes. The first class of the hematopoetic superfamily includes receptors for human growth hormone (hGH), erythropoietin, garanulocyto-macrophage colony-stimulating factors, interleukin-3, -4, -6 and -7 while the second class of the cytokine receptor family included the receptors for IFN-γ, IFN-α and IL-10. The structures of hGHR and tissue factor belong to class 1 and class 2 of cytokine receptor superfamily, respectively, and have been well characterized by mutational analysis and crystal structure analysis. The main difference in these two receptors is the angle between domains. The angle between domains in hGHR is about 85° while that of tissue factor is about 120°. The known structure of tissue factor was used as a backbone to construct a computer model of the ECD of hIFNAR2 (FIG. 3). The ECD of hIFNAR2 is composed of 2 domains (approximately 100 amino acids/ domain) while the ECD of hIFNAR1 is composed of 4 domains. The multiple alanine substitutions in residues of 49-54, 68-72, 74-78, 105-109, 133-139 and 153-156 of IFNAR2 completely abolished the binding of IFN-α 2/1 as well as mAb 1 F3 to IFNAR2. Upon a comparison of the computer model and the location of the residues identified in the multiple mutant analysis, it is apparent that these residues collectively form a patch. Accordingly, these residues play an important role in the binding of the ligand and mAb 1F3. Single alanine mutant analysis showed that residue D68, E78 and W101 are crucial in the binding of IFN-α 2/1 while residues I104 and D105 contribute to the binding of IFN-α 2/1. Since the residues D68, E78, W101, 1104 and D105 form a small pocket in the computer model, the data indicate that this pocket is important in binding to the ligand. Although the portion of IFNAR2 involved in the binding of mAb 1F3 exhibits a significant overlap with the portion of IFNAR2 involved in the binding of IFN-α 2/1 as determined by the multiple alanine mutation analysis, no single residue was shown to be crucial for the binding of mAb 1F3 binding whereas residues D68, E78 and W101 were shown to be crucial for the binding of IFN-α 2/1. This result indicates that the binding of blocking mAb 1 F3 to the receptor is not necessarily same as the binding of ligand to the receptor.

The epitopes recognized by mAb 3B7 and mAb 1 D3 were localized to domain 1 (residues 49-55 and 68-72 in particular) and domain 2 (residues 133-139 and 153-157 in particular) IFNAR2, respectively. Although, the epitope(s) recognized by mAb 3B7 do not closely overlap with the epitope recognized by IFN-α 2/1, mAb 3B7 exhibited the most potent blocking activity. In contrast, mAb 1D3 recognized residues located in lower domain 2 of IFNAR2, which forms a part of the ligand binding areas and showed weak blocking activities in the ISGF complex formation and in anti-viral assay.

The following hybridomas have been deposited with the American Type Culture Collection, 12301 Parklawn Drive, Rockville, Md., USA (ATCC):

| Cell Lines | ATCC Accession No. | Deposit Date |
|---|---|---|
| 3B7 | | |
| 1F3 | | |
| 1D3 | | |

These deposits were made under the provisions of the Budapest Treaty on the International Recognition of the Deposit of Microorganisms for the Purpose of Patent Procedure and the Regulations thereunder (Budapest Treaty). This assures maintenance of a viable deposit for 30 years from the date of deposit. These cell lines will be made available by ATCC under the terms of the Budapest Treaty, and subject to an agreement between Genentech, Inc. and ATCC, which assures permanent and unrestricted availability of the cell lines to the public upon issuance of the pertinent U.S. patent or upon laying open to the public of any U.S. or foreign patent application, whichever comes first, and assures availability of the cell lines to one determined by the U.S. Commissioner of Patents and Trademarks to be entitled thereto according to 35 USC §122 and the Commissioner's rules pursuant thereto (including 37 CFR §1.14 with particular reference to 886 OG 638).

The assignee of the present application has agreed that if the deposited cell lines should be lost or destroyed when cultivated under suitable conditions, they will be promptly replaced on notification with a specimen of the same cell line. Availability of the deposited cell lines is not to be construed as a license to practice the invention in contravention of the rights granted under the authority of any government in accordance with its patent laws.

What is claimed is:

1. An anti-IFNAR2 antibody selected from the group consisting of antibody 1F3 poduced by a hybridoma with ATCC Accession No. HP 12426 or progeny thereof, antibody 3B7 produced by a hybridoma with ATCC Accession No. HP 12427 or progeny thereof, and antibody 1D3 produced by a hybridoma with ATCC Accession No. HP 12428 or progeny thereof.

2. An anti-IFNAR2 antibody that competes for binding to IFNAR2 with the antibody of claim 1.

3. A polypeptide comprising a portion of the antibody of claim 1 or 2, wherein said portion comprises an antigen binding or a variable region of said antibody.

4. The polypeptide of claim 3, wherein said portion comprises at least one complementary determining region of said antibody.

5. The antibody of claim 2, wherein said antibody does not substantially block binding of a Type I interferon to IFNAR2.

6. The antibody of claim 5, wherein said Type I interferon is IFNα-2/1.

7. The antibody of claim 5, wherein said antibody blocks anti-viral activity of a first Type I interferon and does not block anti-viral activity of a second Type I interferon.

8. The antibody of claim 5, wherein said antibody competes for binding to IFNAR2 with antibody 1D3.

9. The antibody of claim 1 or 2, wherein said antibody is a monoclonal antibody.

10. The antibody of claim 1 or 2, wherein said antibody is a humanized antibody.

11. The antibody of claim 1 or 2, wherein said antibody is a human antibody.

12. A method of treating an immune-mediated disorder in a subject comprising administering to the subject the antibody of claim 1 or 2.

13. The method of claim 12, wherein said immune-mediated disorder is selected form the group consisting of type I diabetes, type II diabetes, systemic lupus erythematosis and rheumatoid arthritis.

14. A composition comprising the antibody of claim 1 or 2 and an excipient.
